SEQUENCE LISTING (25 sequences)

SEQ ID NO: 1 - protein, 139 aa, Clostridium perfringens
AIIETAIPQSEMTASATSEEGQDPASSAIDGNTNTMWHTKWNGSDALPQSLSVNLGSSRKVSSIAITPRTSGNNGFITKYEIHAINNGVETLVAEGTWEENNLVKTVTFDSPIDAEEIKITAIQGVGGFASIAELNVYE

SEQ ID NO: 2 - protein, 1173 aa, Clostridium perfringens
MKSKKIIATLVASLVISNMGGYLVKANPNVNHKAVIIEDRQAIIETAIPQSEMTASATSEEGQDPASSAIDGNTNTMWHTKWNGSDALPQSLSVNLGSSRKVSSIAITPRTSGNNGFITKYEIHAINNGVETLVAEGTWEENNLVKTVTFDSPIDAEEIKITAIQGVGGFASIAELNVYEIKGEVDEIANYGNLKITKEEERLNITRDLEKFSSLDEGTIVTRFNMNDTSIQSLIGLSDGNKANNYFSLYVSGGKVGYELRRQEGNGDFNVHHSADVTFNKGINTLALKIEKGVGAKIFLNGSLVKTVSDPNIKFLNAINLNSGFIGKTDRANGYNEYLFRGNIDFMNIYDKPVSDNYLLRKTGETKAPSEDSLLPDDVYKTQPVELFYPGYLESRGYRIPALETTKKGTVLASIDVRNNGDHDAPNNNIDVGIRRKEVNGEWEEGKVILDYPGKSAAIDTSLMSATIEENGIEKERIFLIVTHFPEGYGFPNTEGGSGYKEIDGKYYFILKDAQNNEYTVREDGIVYNSEGNETDYVMKNDKTLIQNGEEVGNALLSNSPLKAVGTAHIEMIYSDDDGNTWSEPEDLNPGLKKEWMKFFGTAPGKGIQIKNGEHKGRLVFPIYYTNQNNFQSSAVIYSDDFGETWKLGESPIDTASVSSETVSSGTQLTECQVVEMPNGQLKLFMRNTGSYTRIATSFDGGATWHDEVPEDTSLREPYCQLSVINYSGKINGKDAIIFSNPDASSRVNGSVKVGLINENGTYENGQPRYEFDWIYNKTVKPGSFAYSCLTELPDGNLGLFYEGEGAGRMAYTEFDLNYLKFNASEDSPAATVQSIESLDEDLIYNAGDEVSIKVNFNQLVSLIGDRKITLDIGGVDVPLNMVNYEGKSSAIFKGTIPEGINPGNYEIKLKENNALELNTVYNKVSTLNGLDNTGINVQIGELKTTVGNSTIKVNEEVQVGSAFEAILGIKGLNGDTEVYSAEYLFEYNAEAFKLNEITSFSDSLFVKSKEVEPGKVRILVASLGNEIEKDSELVKVNLTPKISSELEVLGLTTALVGAGDGNTHDLELSSKEVKINEEASGEIVVNPVQNFEIPEINKKNVKLTWNAPITTEGLEGYVIYKDGKKLSEVPAESTEFVVSKLNRHTIYNFKVAAKYSNGELSAKESKTIRTAR

SEQ ID NO: 3 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 4 - protein, 192 aa, Vibrio cholerae
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 5 - protein, 1035 aa, Streptococcus pneumoniae
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 6 - protein, 185 aa, Streptococcus pneumoniae
VIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 7 - protein, 145 aa, Streptococcus pneumoniae
TPDKFNDGNLNIAYAKPTTQSSVDYNGDPNRAVDGNRNGNFNSGSVTHTRADNPSWWEVDLKKMDKVGLVKIYNRTDAETQRLSNFDVILYDNNRNEVAKKHVNNLSGESVSLDFKEKGARYIKVKLLTSGVPLSLAEVEVFRES

SEQ ID NO: 8 - protein, 896 aa, Streptococcus pneumoniae
MNKEKIKRKLITILFVCIGMLCFGLLAGVKADNRVQMRTTINNESPLLLSPLYGNDNGNGLWWGNTLKGAWEAIPEDVKPYAAIELHPAKVCKPTSCIPRDTKELREWYVKMLEEAQSLNIPVFLVIMSAGERNTVPPEWLDEQFQKYSVLKGVLNIENYWIYNNQLAPHSAKYLEVCAKYGAHFIWHDHEKWFWETIMNDPTFFEASQKYHKNLVLATKNTPIRDDAGTDSIVSGFWLSGLCDNWGSSTDTWKWWEKHYTNTFETGRARDMRSYASEPESMIAMEMMNVYTGGGTVYNFECAAYTFMTNDVPTPAFTKGIIPFFRHAIQNPAPSKEEVVNRTKAVFWNGEGRISSLNGFYQGLYSNDETMPLYNNGRYHILPVIHEKIDKEKISSIFPNAKILTKNSEELSSKVNYLNSLYPKLYEGDGYAQRVGNSWYIYNSNANINKNQQVMLPMYTNNTKSLSLDLTPHTYAVVKENPNNLHILLNNYRTDKTAMWALSGNFDASKSWKKEELELANWISKNYSINPVDNDFRTTTLTLKGHTGHKPQINISGDKNHYTYTENWDENTHVYTITVNHNGMVEMSINTEGTGPVSFPTPDKFNDGNLNIAYAKPTTQSSVDYNGDPNRAVDGNRNGNFNSGSVTHTRADNPSWWEVDLKKMDKVGLVKIYNRTDAETQRLSNFDVILYDNNRNEVAKKHVNNLSGESVSLDFKEKGARYIKVKLLTSGVPLSLAEVEVFRESDGKQSEEDIDKITEDKVVSTNKVATQSSTNYEGVAALAVDGNKDGDYGHHSVTHTKEDSPSWWEIDLAQTEELEKLIIYNRTDAEIQRLSNFDIIIYDSNDYEVFTQHIDSLESNNLSIDLKGLKGKKVRISLRNAGIPLSLAEVEVYTYK

SEQ ID NO: 9 - protein, 165 aa, Streptomyces avermitilis
APSLEGSSWIWFPEGEPANSAPAATRWFRRTVDLPDDITGATLAISADNVYAVSVDGAEVARTDLEADNEGWRRPAVIDVLDHVHSGNNTLAVSASNASVGPAGWICVLVLTTASGEKKIFSDASWKSTDHEPADGWREPDFDDSGWPAAKVAAAWGAGPWGRVA

SEQ ID NO: 10 - protein, 1030 aa, Streptomyces avermitilis
MSALRVTSPSVEYVQRPLGLDAAHPRLSWPMASAAPGRRQSAYQVRVASSAAGLSHPDVWDSGKVVSDDSVLVPYAGPPLKPRTRYFWSVRVWDADGGASEWSAPSWWETGLMGASQWSAKWISAPAPLTEAPSLEGSSWIWFPEGEPANSAPAATRWFRRTVDLPDDITGATLAISADNVYAVSVDGAEVARTDLEADNEGWRRPAVIDVLDHVHSGNNTLAVSASNASVGPAGWICVLVLTTASGEKKIFSDASWKSTDHEPADGWREPDFDDSGWPAAKVAAAWGAGPWGRVAPVASAANQLRHEFRLPHKKVSRARLYATALGLYEAHLNGRRVGRDQLAPGWTDYRKRVQYQTYDVTSSVRPGANALAAYVAPGWYAGNVGMFGPHQYGERPALLAQLEVEYADGTSERITSGPDWRAASGPIVSADLLSGETYDARKETAGWTSPGFDDRAWLAVRGADNDVPEQIVAQVDGPVRIAKELPARKVTEPKPGVFVLDLGQNMVGSVRLRVSGDAGTTVRLRHAEVLNPDGTIYTANLRSAAATDTYTLKGQGEETYEPRFTFHGFRYVEVTGFPGKPSTTSVTGRVMHTSAPFTFEFETNVPMLNKLHSNITWGQRGNFLSVPTDTPARDERLGWTGDINVFAPTAAYTMESARFLTKWLVDLRDAQTSDGAFTDVAPAVGNLGNGVAGWGDAGVTVPWALYQAYGDRQVLADALPSVHAWLRYLEKHSDGLLRPADGYGDWLNVSDETPKDVIATAYFAHSADLAARMATELGKDAAPYTDLFTRIRKAFQTAYVASDGKVKGDTQSAYVLTLSMNLVPDALRKAAADRLVALIEAKDWHLSTGFLGTPRLLPVLTDTGHTDVAYRLLHQRTFPSWGYPIDKGSTTMWERWDSIQPDGGFQTPEMNSFNHYAYGSVGEWMYANIAGIAPGRAGYRQVVIRPRPGGEVTSARATFASLHGPVSTRWQQRSGGFVLTCSVPPNTTAEVWIPADHPDRVQHTHGTFVRAEDGCAVFEVGSGSHRFTV

SEQ ID NO: 11 - protein, 159 aa, Streptococcus pneumoniae
NLVENGDFGQTEDGSSPWTGSKAQGWSAWVDQKNSADASTRVIEAKDGAITISSHEKLRAALHRMVPIEAKKKYKLRFKIKTDNKIGIAKVRIIEESGKDKRLWNSATTSGTKDWQTIEADYSPTLDVDKIKLELFYETGTGTVSFKDIELVEVADQLS

SEQ ID NO: 12 - protein, 1066 aa, Streptococcus pneumoniae
MQTKTKKLIVSLSSLVLSGFLLNHYMTIGAEETTTNTIQQSQKEVQYQQRDTKNLVENGDFGQTEDGSSPWTGSKAQGWSAWVDQKNSADASTRVIEAKDGAITISSHEKLRAALHRMVPIEAKKKYKLRFKIKTDNKIGIAKVRIIEESGKDKRLWNSATTSGTKDWQTIEADYSPTLDVDKIKLELFYETGTGTVSFKDIELVEVADQLSEDSQTDKQLEEKIDLPIGKKHVFSLADYTYKVENPDVASVKNGILEPLKEGTTNVIVSKDGKEVKKIPLKILASVKDAYTDRLDDWNGIIAGNQYYDSKNEQMAKLNQELEGKVADSLSSISSQADRTYLWEKFSNYKTSANLTATYRKLEEMAKQVTNPSSRYYQDETVVRTVRDSMEWMHKHVYNSEKSIVGNWWDYEIGTPRAINNTLSLMKEYFSDEEIKKYTDVIEKFVPDPEHFRKTTDNPFKALGGNLVDMGRVKVIAGLLRKDDQEISSTIRSIEQVFKLVDQGEGFYQDGSYIDHTNVAYTGAYGNVLIDGLSQLLPVIQKTKNPIDKDKMQTMYHWIDKSFAPLLVNGELMDMSRGRSISRANSEGHVAAVEVLRGIHRIADMSEGETKQCLQSLVKTIVQSDSYYDVFKNLKTYKDISLMQSLLSDAGVASVPRPSYLSAFNKMDKTAMYNAEKGFGFGLSLFSSRTLNYEHMNKENKRGWYTSDGMFYLYNGDLSHYSDGYWPTVNPYKMPGTTETDAKRADSDTGKVLPSAFVGTSKLDDANATATMDFTNWNQTLTAHKSWFMLKDKIAFLGSNIQNTSTDTAATTIDQRKLESGNPYKVYVNDKEASLTEQEKDYPETQSVFLESFDSKKNIGYFFFKKSSISMSKALQKGAWKDINEGQSDKEVENEFLTISQAHKQNRDSYGYMLIPNVDRATFNQMIKELESSLIENNETLQSVYDAKQGVWGIVKYDDSVSTISNQFQVLKRGVYTIRKEGDEYKIAYYNPETQESAPDQEVFKKLEQAAQPQVQNSKEKEKSEEEKNHSDQKNLPQTGEGQSILASLGFLLLGAFYLFRRGKNN

SEQ ID NO: 13 - protein, 438 aa, Pseudomonas aeruginosa
MNTYFDIPHRLVGKALYESYYDHFGQMDILSDGSLYLIYRRATEHVGGSDGRVVFSKLEGGIWSAPTIVAQAGGQDFRDVAGGTMPSGRIVAASTVYETGEVKVYVSDDSGVTWVHKFTLARGGADYNFAHGKSFQVGARYVIPLYAATGVNYELKWLESSDGGETWGEGSTIYSGNTPYNETSYLPVGDGVILAVARVGSGAGGALRQFISLDDGGTWTDQGNVTAQNGDSTDILVAPSLSYIYSEGGTPHVVLLYTNRTTHFCYYRTILLAKAVAGSSGWTERVPVYSAPAASGYTSQVVLGGRRILGNLFRETSSTTSGAYQFEVYLGGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 14 - protein, 106 aa, Pseudomonas aeruginosa
VPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 15 - protein, 489 aa, Streptococcus pneumoniae
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGALGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 16 - protein, 489 aa, Artificial Sequence
  CBM
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 17 - protein, 489 aa, Artificial Sequence
  CBM
GAMVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 18 - protein, 5 aa, Artificial Sequence
  Linker
ALNGS

SEQ ID NO: 19 - protein, 5 aa, Artificial Sequence
  Linker
LQALG

SEQ ID NO: 20 - protein, 5 aa, Artificial Sequence
  Linker
GGNSG

SEQ ID NO: 21 - protein, 5 aa, Artificial Sequence
  Linker
GGGSG

SEQ ID NO: 22 - protein, 5 aa, Artificial Sequence
  Linker
GGALG

SEQ ID NO: 23 - protein, 10 aa, Artificial Sequence
  Linker
ALNGSGGGSG

SEQ ID NO: 24 - protein, 10 aa, Artificial Sequence
  Linker
LQALGGGGSL

SEQ ID NO: 25 - protein, 15 aa, Artificial Sequence
  Linker
ALNGSGGGSGGGGSG

(19) United States
(12) Patent Application Publication
(10) Pub. No.: US 2023/0125561 A1
(43) Pub. Date: Apr. 27, 2023

(54) TREATMENT OF VIRAL INFECTION

(71) Applicant: Pneumagen Limited, Fife (GB)

(72) Inventors: Garry Taylor, Fife (GB); Helen Connaris, Fife (GB); Jane Potter, Fife (GB); Graeme Rogers, Fife (GB); Angus Aitken, Fife (GB); Antoni Tortajada, Fife (GB); Douglas Thomson, Fife (GB); Lei Yang, Fife (GB)

(21) Appl. No.: 17/905,303

(22) PCT Filed: Mar. 4, 2021
(86) PCT No.: PCT/EP2021/055515

§ 371 Date: Aug. 30, 2022

(30) Foreign Application Priority Data
Mar. 5, 2020 (GB) 2003232.2
Apr. 4, 2020 (GB) 2004998.7
Apr. 27, 2020 (GB) 2006160.2

(51) Int. Cl. A61K 38/16; A61P 31/14

(52) U.S. Cl. CPC A61K 38/164; A61P 31/14

(57) ABSTRACT

Disclosed are molecules useful in the treatment or prevention of viral infections in humans and animals and/or the treatment or prevention of the associated symptoms, diseases and/or conditions. The disclosure provides molecules which comprise sugar (carbohydrate/polysaccharide/sialic acid/glycan)-binding protein(s) which are useful in the treatment or prevention of Coronavirus infections and/or diseases, symptoms and/or conditions caused or contributed to by the same.

### RELATED APPLICATIONS

This application is a 35 U.S.C. § 371 national stage of PCT Application No. PCT/EP2021/055515, filed on Mar. 4, 2021, which claims priority from United Kingdom Patent Application Nos. 2003232.2, 2004998.7, and 2006160.2, filed on Mar. 5, 2020, Apr. 4, 2020, and Apr. 27, 2020, respectively, the contents of which are incorporated herein by reference. The above-referenced PCT International Application was published in the English language as International Publication No. WO 2021/176017 A1 on Sep. 10, 2021.

### FIELD OF THE INVENTION

The disclosure provides molecules for use in compositions, medicaments and methods for the treatment or prevention of viral diseases and/or conditions.

### BACKGROUND

Viral pathogens can cause an array of diseases and/or conditions in humans and while vaccines and antiviral therapies are available for use against some pathogens, for others there are no useful therapeutic options. In some cases, the treatment of a viral infection is limited to patient isolation (to minimise the risk of further spread) and treatment of the symptoms. In some cases a viral pathogen, in particular a respiratory viral pathogen, can lead to secondary complications such as shortness of breath, pneumonia, bronchitis and/or bronchiolitis. The young, old, immunocompromised, pregnant and individuals with underlying health conditions are often most at risk from viral pathogens.

The pathology of any given viral pathogen will vary, but those causing respiratory illness will most often be transmitted by aerosolised droplets generated by, for example, coughs and sneezes and fluids/secretions from/to mucus membranes (including the respiratory airways, the lungs, nose, mouth and eyes).

Occasionally, a viral outbreak concerns a species and/or strain of pathogen that is new to science. There is often no effective treatment for these infections and outbreaks must be contained and controlled to prevent uncontrolled spread.

Severe acute respiratory syndrome (SARS) is a viral respiratory disease of zoonotic origin. This disease is caused by the SARS Coronavirus and the symptoms may include a fever and/or ‘flu’-like illness with muscle pain, a cough, sore throat and other nonspecific symptoms (including, for example, lethargy/malaise). Infection with the SARS Coronavirus may also result in a shortness of breath, viral pneumonia and/or secondary bacterial pneumonia.

Vaccines are often difficult to develop, taking a considerable time to prepare and test. Also, antiviral drugs are variably effective and, in many cases, the virus infection can resolve before the drug takes effect.

Accordingly, there is a need for new and effective treatments to combat viral infections, in particular viral respiratory infections.

### SUMMARY

Disclosed herein are molecules which may be used in the treatment or prevention of viral infections in humans and animals and/or the treatment or prevention of the associated diseases and/or conditions.

The described molecules may find particular application in the treatment or prevention of viral respiratory pathogens, including for example pathogens belonging to the Family Coronaviridae.

It should be noted that the terms “comprise”, “comprising” and/or “comprises” is/are used to denote that the various aspects and embodiments of this disclosure “comprise” a particular feature or features. It should be understood that this/these terms may also encompass aspects and/or embodiments which “consist essentially of” or “consist of” the relevant feature or features.

The Coronaviridae are a group of enveloped, positive-sense, single-stranded RNA viruses. The Coronaviridae contain the genus Coronavirus. The name “Coronaviridae” or “Coronavirus” is derived from the distinctive shape of the virus which contains a number of crown-like projections (“peplomers” or “spikes”). Coronaviruses cause respiratory tract infections in humans and outbreaks of deadly pneumonia worldwide.

The Coronaviruses are large enveloped, positive strand RNA viruses that have been classified into 4 genera: Alpha-, Beta-, Gamma-, and Deltacoronavirus. They have the largest genome across all RNA viruses (27-32 kb), packed inside a helical capsid surrounded by an envelope. There are at least 3 structural proteins associated with the viral envelope: the membrane protein (M), the envelope protein (E) and the spike (glyco)protein (S). Some Coronavirus encode a hemagglutinin-esterase protein (HE).

The S glycoprotein forms large protrusions on the viral surface (forming the “corona” or “crown”) and it is involved in viral entry to the host cell. The S protein has a large ectodomain divided into the following domains/regions: S1 (receptor binding domain); S2 (membrane fusion domain); a transmembrane anchor; and a short intracellular tail. The S1 domain is divided into 2 major domains: (i) N-terminal domain (S1-NTD)—responsible for binding sugar; and (ii) the C-terminal domain (S1-CTD)—responsible for recognizing protein receptors ACE2, APN, and DPP4.

As used herein, the term “Coronavirus” embraces any virus classed as belonging to the Family Coronaviridiae and embraces the SARS Coronavirus; the MERS Coronavirus; and SARS-CoV-2 Coronavirus.

The term “Coronavirus” also embraces all SARS-CoV-2 variants. For example, the term “Coronavirus” embraces variants B.1.1.7, B.1525, B.1.351 and the variant referred to as P1. These variants may be characterised by mutations (for example amino acid additions, substitutions and/or deletions) within the spike protein. For example, the term “Coronavirus” may embrace any variant with one or more of the following mutations within the spike protein:
(i) HV69-70 deletion; and/or
(ii) N501Y; and/or
(iii) E484K.

In all cases, a variant spike protein sequence may contain one or more mutations relative to a reference sequence. A suitable reference sequence may be the Wuhan-Hu-1 strain, S-proteins sequences from which are available under accession codes QHD43416.1/YP_009724390.1.

SARS-CoV-2 is classified as belonging to the same genus as both SARS and MERS (Betacoronavirus); it is in the same sub-genus grouping as SARS, sharing around 80% nucleotide identity across the whole genome. Both SARS and SARS-CoV-2 use the glycosylated ACE2 protein expressed on the host cell surface for cell entry. Glycosylation of ACE2 residue 90 has been shown to significantly inhibit the SARS virus. The SARS S protein has a predicted 21 glycosylation sites, with at least 18 of these residues being conserved in the SARS-CoV-2 sequence.

Without wishing to be bound by any particular theory, antibodies which neutralise Coronavirus (for example, the MERS Coronavirus) may target the receptor-binding domain (RBD) of the spike glycoprotein and block its binding to the cellular receptor dipeptidyl peptidase 4 (DPP4). Other (anti-S protein N-terminal domain (NTD)) antibodies have been shown to bind to the N-terminal domain (NTD) of the spike glycoprotein and inhibit host cell entry with high potency. Thus, by targeting the NTD part of the S-protein, it may be possible to inhibit host cell entry in a way which is not dependent on the RBD (S1-CTD) site; that inhibition may affect the conformational state of the S glycoprotein. It is also suggested that, like certain types of influenza (for example, influenza type C), Coronavirus can bind host cell sialic acid containing receptors—in particular sialoglycans, including, for example, cell surface components containing 9-O-acetylated sialic. Moreover (and again, without wishing to be bound by theory), Coronaviruses possess a glycoprotein profile that might be recognised by a variety of CBMs including those classified as belonging to CBM families 32, 40, 47, 67 and 70.

Thus, proteins with an affinity for certain carbohydrates (polysaccharides or glycans), including, for example, sialic acid (e.g. any of the CBM(s) as described herein) have the potential to disrupt the infectivity of Coronavirus, including SARS-CoV-2, by targeting glycans on the host and on the virus.

Disclosed are a number of molecules which comprise (consist of, or consist essentially of) sugar (carbohydrate/polysaccharide/glycan)-binding protein(s). Proteins of this type exhibit a particular affinity for glycans and/or sialic acid and are useful in the treatment or prevention of Coronavirus infections and/or diseases and/or conditions caused or contributed to by the same.

The phrase “disease or condition caused or contributed to by a Coronavirus” may include those diseases or conditions referred to as SARS and MERS and/or any other respiratory disease and/or condition associated with a Coronavirus infection. Additionally, the phrase “disease or condition caused or contributed to by a Coronavirus” includes the acute respiratory disease referred to as COVID-19 and which is caused by, or which is associated with, a SARS-CoV-2 infection.

As used herein, the term treatment may embrace a reduction in one or more of the symptoms associated with a disease/condition caused or contributed to by a Coronavirus infection. Such symptoms may include, for example, a (continuous) cough, a fever, change/loss in/of taste/smell. Accordingly, any of the glycan binding molecules described herein may be used in compositions and or methods for the treatment of one or more of the symptoms of a Coronavirus infection. By way of example, any one of the glycan binding molecules of this invention may be used to reduce the continuous cough that might develop as a consequence of a Coronavirus infection.

In view of the above, this disclosure provides a glycan binding molecule for use in the treatment or prevention of a Coronavirus infection and/or a disease or condition caused or contributed by a Coronavirus.

Also disclosed is a method of treating or preventing a Coronavirus infection and/or a disease or condition caused or contributed by a Coronavirus, said method comprising administering a subject in need thereof a glycan binding molecule.

Provided herein is the use of a glycan binding molecule in the manufacture of a medicament for the treatment or prevention of prevention of a Coronavirus infection and/or a disease or condition caused or contributed by a Coronavirus.

This disclosure also provides a sialic acid binding molecule for use in the treatment or prevention of a Coronavirus infection and/or a disease or condition caused or contributed by a Coronavirus.

Also disclosed is a method of treating or preventing a Coronavirus infection and/or a disease or condition caused or contributed by a Coronavirus, said method comprising administering a subject in need thereof a sialic acid binding molecule.

Provided herein is the use of a sialic acid binding molecule in the manufacture of a medicament for the treatment or prevention of prevention of a Coronavirus infection and/or a disease or condition caused or contributed by a Coronavirus.

A particularly useful molecule is the carbohydrate-binding module (CBM). Thus, a carbohydrate, glycan and/or sialic acid binding molecule for the uses, methods and medicaments described herein may be, or may comprise, a CBM.

Accordingly, the disclosure further provides:
(i) A CBM for use in the treatment or prevention of a Coronavirus infection and/or a disease or condition caused or contributed by a Coronavirus;
(ii) A method of treating or preventing a Coronavirus infection and/or a disease or condition caused or contributed by a Coronavirus, said method comprising administering a subject in need thereof a CBM.
(iii) Use of a CBM in the manufacture of a medicament for the treatment or prevention of prevention of a Coronavirus infection and/or a disease or condition caused or contributed by a Coronavirus.

Based on the above, this disclosure provides a CBM for use in the treatment or prevention of a COVID-19 infection and/or a disease or condition caused or contributed by SARS-CoV-2.

The disclosure also provides a method of treating or preventing a COVID-19 infection and/or a disease or condition caused or contributed to by SARS-CoV-2, said method comprising administering a subject in need, thereof a CBM.

Described herein is the use of a CBM in the manufacture of a medicament for the treatment or prevention of prevention of a COVID-19 infection and/or a disease or condition caused or contributed by SARS-CoV-2.

Examples of useful CBMs are provided below, but for completeness it should be understood that the term “CBM” includes, for example, CBMs classified as belonging to CBM families 32, 40, 47, 67 and 70. One of skill will appreciate that there is a vast array of different CBMs and members of the abovementioned families can be derived from many different bacterial species Further information regarding CBMs, in particular the family 32, 40, 47, 67 and 70 CBMs, can be found at the public CAZY database (available at: www.cazy.org). Some particularly useful CBMs may be derived from bacterial species within the Genera: Streptococcus, Vibrio and Clostridium. It should also be understood (and as is set out in more detail below) the disclosure embraces the use of molecules which comprise one or more CBMs and modified forms of any of the CBMs described herein. A modified CBM is a CBM which includes one or more mutated residues relative to the wild-type CBM sequence.

The CBMs for the various uses, medicaments and methods described herein are all types of carbohydrate, glycan and/or sialic acid binding molecule.

The term “sialic acid” embraces all forms of N- or O-substituted neuraminic acid and includes all synthetic, naturally occurring and/or modified forms thereof. Sialic acids may be found as components of cell surface molecules, glycoproteins and glycolipids. Most often, sialic acids are present at the end (terminal regions) of sugar chains connected to cell membranes and/or proteins. For example, some cells of the human upper respiratory tract comprise α-2,6-linked sialic acid receptors and other cells of the upper and lower respiratory tracts comprise α-2,3-linked sialic acid receptors. The sialic acid family encompasses a number (approximately 50) of derivatives that may result from acetylation, glycolylation, lactonisation and methylation at C4, C5, C7, C8 and C9. All such derivatives are to be embraced by the term “sialic acid”. Sialic acids may be found linked α(2,3) or α(2,6) to Gal and GaINAc or α(2,8) or α(2,9) to another sialic acid. Accordingly, it is important to understand that while the term “sialic acid” is used throughout this specification, it encompasses all anomers, derivatives, analogues or variants (either naturally occurring or synthetically generated) thereof as well as all monomers, dimers, trimers, oligomers, polymers or concatamers comprising the same.

Accordingly, a sialic acid binding molecule for the various methods, uses and medicaments of this disclosure may comprise a moiety with affinity for sialic acid in any of its various forms as described above. Indeed, a CBM for use, or a CBM for use in a medicament or method described herein, may exhibit an affinity for sialic acid in any of its various forms as described above and/or may bind/couple to and/or associate with sialic acid molecules as may be present in or on, a Coronavirus, a (mammalian) cell surface and/or a (mammalian) cell surface receptor.

Useful CBMs may take any form and/or belong to any class or type of CBM. CBMs from any one or more of the following CBM families, may be of use.
(i) Family 32 CBMs (CBM32);
(ii) Family 40 CBMs (CBM40);
(iii) Family 47 CBMs (CBM47);
(iv) Family 67 CBMs (CBM67); and
(v) Family 70 CBMs (CBM70).

For example, the disclosure provides a CBM32 for use in the treatment or prevention of:
(i) a Coronavirus infection and/or a disease or condition caused or contributed by a Coronavirus; or
(ii) a COVID-19 infection and/or a disease or condition caused or contributed by SARS-CoV-2.

Also disclosed is a method of treating or preventing:
(i) a Coronavirus infection and/or a disease or condition caused or contributed by a Coronavirus; or
(ii) a COVID-19 infection and/or a disease or condition caused or contributed by SARS-CoV-2;
said method comprising administering a subject in need thereof one or more Family 32 CBM32.

Additionally disclosed, is the use of a CBM32 in the manufacture of a medicament for the treatment or prevention of a Coronavirus infection and/or a disease or condition caused or contributed by a Coronavirus.

This disclosure also provides a CBM40, or a CBM47, or a CBM67 or a CBM70 for use in the treatment or prevention of:
(i) a Coronavirus infection and/or a disease or condition caused or contributed by a Coronavirus; or
(ii) a COVID-19 infection and/or a disease or condition caused or contributed by SARS-CoV-2.

The disclosure further relates to a method of treating or preventing:
(i) a Coronavirus infection and/or a disease or condition caused or contributed by a Coronavirus; or
(ii) a COVID-19 infection and/or a disease or condition caused or contributed by SARS-CoV-2;
said method comprising administering a subject in need thereof one or more Family 40 CBM, a Family 47 CBM, a Family 67 CBM or a Family 70 CBM.

Additionally, the disclosure provides the use of a CBM40, or a CBM47, or a CBM67 or a CBM70 in the manufacture of a medicament for the treatment or prevention of prevention of a Coronavirus infection and/or a disease or condition caused or contributed by a Coronavirus.

It should be noted that the use of any one of the disclosed CBM32s, CBM40s, CBM47s, CBM67s or CBM70s, may be combined with any other carbohydrate binding protein, glycan binding protein, sialic acid binding molecule and/or CBM. Indeed, the use of a CBM32, a CBM40, a CBM47, a CBM67 or a CBM70 may be combined with the use of any other one of a CBM32, a CBM40, a CBM47, a CBM 67 or a CBM70.

Further detail concerning each CBM family is provided below—all of these CBMs (their sub-types, variants, orthologues etc.) are for use in the treatment and/or prevention of Coronavirus infections and/or Coronavirus associated diseases and/or conditions described herein.

CBM32

A useful CBM32 (i.e. a CBM32 for the various methods, medicaments and uses described herein) may be derived from any suitable source. For example, CBM32s for use may be obtained from microorganisms, including, for example, bacteria of the genera Cellvibrio, Yersinia, Micromonospora, Streptococcus, Bifidobacteria and Clostridium. For example, useful CBM32s may be obtained or derived from, for example, Cellvibrio mixtus, Yersinia enterolitica, Clostridium perfringens, Clostridium thermocellum, Streptococcus pneumoniae, Bifidobacterium longum and Micromonospora viridifaciens. Further details concerning the source, structure and function of the CBM32 family can be found within the Carbohydrate Active Enzymes database (freely available on the internet at: www.cazy.org/CBM32.html).

An exemplary CBM32 sequence is provided by SEQ ID NO: 1 below:

SEQ ID NO: 1
AIIETAIPQSEMTASATSEEGQDPASSAIDGNTNTMWHTKWNGSDALP
QSLSVNLGSSRKVSSIAITPRTSGNNGFITKYEIHAINNGVETLVAEG
TWEENNLVKTVTFDSPIDAEEIKITAIQGVGGFASIAELNVYE

Accordingly, a CBM for use may comprise, consist essentially or consist of a CBM having the sequence of SEQ ID NO: 1 or a carbohydrate binding portion thereof. A carbohydrate binding fragment of SEQ ID NO: 1 may comprise anywhere between about 5, 6, 7, 8, 9 or 10 (consecutive or contiguous) amino acids to about 138 (consecutive or contiguous) amino acids from SEQ ID NO: 1. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130 or about 135 (consecutive or contiguous) amino acids from SEQ ID NO: 1.

CBM32 or a protein comprising, consisting essentially or consisting of SEQ ID NO: 1 may bind, for example, galactose, N-acetylgalactosamine (GalNAc), N-acetylglucosamine (GlcNAc) and/or lactose. Accordingly, any fragment for use may also bind galactose, N-acetylgalactosamine (GalNAc), N-acetylglucosamine (GlcNAc) and/or lactose. One of skill will appreciate that the binding affinity of any given CBM32 molecule may depend on the precise CBM32 subtype; by way of further examples, some CBM32s have shown affinity for a variety of ligands (examples include type II blood group H-trisaccharide (Fucα1-2Gal β1-4GlcNAc), N-acetyl-D-lactosamine (LacNAc), galactose, lacto-N-biose, disaccharide GlcNAc-α-1,4-Gal (which may be referred to as an N-acetylglucosamine linked alpha 1,4 to galactose), and/or GlcNAc). It should also be noted that multiple CBM32 subtypes may be derived from a single organism; these different CBM subtypes may exhibit the same, similar or different binding specificities. For example, Clostridium perfringens contains two sialidases NanJ and NanH; NanJ contains one galactose-specific CBM32; NanH contains four putative CBM32s with different binding selectivity—for example, the CBM32 encoded by NanH binds GlcNAc. As used herein, the term CBM32 embraces all CBM32 variants, derivatives and sub-types.

SEQ ID NO: 1 is derived from the sequence deposited in the UniProt database under ID No: A0A2X2YJF2. This sequence is reproduced as SEQ ID NO: 2 below (SEQ ID NO: 1 appears as residues 42-180—shown in bold in the sequence below).

SEQ ID NO: 2
MKSKKIIATL VASLVISNMG GYLVKANPNV NHKAVIIEDR
QAIIETAIPQ SEMTASATSE EGQDPASSAI DGNTNTMWHT
KWNGSDALPQ SLSVNLGSSR KVSSIAITPR TSGNNGFITK
YEIHAINNGV ETLVAEGTWE ENNLVKTVTF DSPIDAEEIK
ITAIQGVGGF ASIAELNVYE IKGEVDEIAN YGNLKITKEE
ERLNITRDLE KFSSLDEGTI VTRFNMNDTS IQSLIGLSDG
NKANNYFSLY VSGGKVGYEL RRQEGNGDFN VHHSADVTFN
KGINTLALKI EKGVGAKIFL NGSLVKTVSD PNIKFLNAIN
LNSGFIGKTD RANGYNEYLE RGNIDFMNIY DKPVSDNYLL
RKTGETKAPS EDSLLPDDVY KTQPVELFYP GYLESRGYRI
PALETTKKGT VLASIDVRNN GDHDAPNNNI DVGIRRKEVN
GEWEEGKVIL DYPGKSAAID TSLMSATIEE NGIEKERIFL
IVTHFPEGYG FPNTEGGSGY KEIDGKYYFI LKDAQNNEYT
VREDGIVYNS EGNETDYVMK NDKTLIQNGE EVGNALLSNS
PLKAVGTAHI EMIYSDDDGN TWSEPEDLNP GLKKEWMKFF
GTAPGKGIQI KNGEHKGRLV FPIYYTNQNN FQSSAVIYSD
DFGETWKLGE SPIDTASVSS ETVSSGTQLT ECQVVEMPNG
QLKLFMRNTG SYTRIATSFD GGATWHDEVP EDTSLREPYC
QLSVINYSGK INGKDAIIFS NPDASSRVNG SVKVGLINEN
GTYENGQPRY EFDWIYNKTV KPGSFAYSCL TELPDGNLGL
FYEGEGAGRM AYTEFDLNYL KFNASEDSPA ATVQSIESLD
EDLIYNAGDE VSIKVNFNQL VSLIGDRKIT LDIGGVDVPL
NMVNYEGKSS AIFKGTIPEG INPGNYEIK LKENNALELNT
VYNKVSTLNG LDNTGINVQI GELKTTVGNS TIKVNEEVQV
GSAFEAILGI KGLNGDTEVY SAEYLFEYNA EAFKLNEITS
FSDSLFVKSK EVEPGKVRIL VASLGNEIEK DSELVKVNLT
PKISSELEVL GLTTALVGAG DGNTHDLELS SKEVKINEEA
SGEIVVNPVQ NFEIPEINKK NVKLTWNAPI TTEGLEGYVI
YKDGKKLSEV PAESTEFVVS KLNRHTIYNF KVAAKYSNGE
LSAKESKTIR TAR

SEQ ID NOS 1 and 2 are derived from Clostridium perfringens. A CBM for use in the various aspects of this disclosure may comprise one, two, three, four or more CBM32s. A CBM for use may comprise one, two, three, four or more proteins comprising SEQ ID NO: 1 or a carbohydrate binding fragment thereof. One of skill will appreciate that useful CBM32s may comprise sequences which exhibit some degree (for example, 99%, 95%, 90%, 85%, 80%, 75%, 70%, 65% or 60%) of sequence identity or homology with the CBM32 sequences of SEQ ID NOS: 1 and 2. All such variant or divergent sequences are to be embraced within the scope of this disclosure and by the term “CBM32”. Identical and/or homologous CBM32 sequences may have carbohydrate binding function.

CBM40

A useful CBM40 (i.e. a CBM40 for the various methods, medicaments and uses described herein) may be derived from any suitable source. For example, CBM40s for use may be obtained from microorganisms, including, for example, bacteria of the genera Clostridium, Enterococcus, Staphylococcus, Streptococcus and Vibrio. For example, useful CBM40s may be obtained or derived from, for example Clostridium perfringens, Streptococcus pneumoniae and Vibrio cholerae. Further details concerning the source, structure and function of the CBM40 family can be found within the Carbohydrate Active Enzymes database (freely available on the internet at: www.cazy.org/CBM40.html).

The Family 40 CBMs embrace molecules of approximately 200 residues and are often found at the N-terminus of GH33 sialidases. They may also be found inserted in the β-propeller of GH33 sialidases. At least the CBM40 of Vibrio cholerae binds the alpha-anomer of sialic acid and, for example, α(2,3)-, α(2,6)-, and α(2,8)-linked sialosides.

Exemplary CBM40s for use may comprise the sialic acid binding domain of Vibrio cholerae NanH sialidase (VcCBM: a CBM40) and/or the equivalent (or homologous) domain from Streptococcus pneumoniae NanA sialidase (SpCBM: also a CBM40). Of course, similar or homologous sialic acid binding modules present in other organisms are to be encompassed within the scope of the terms “CBM” and “CBM40”.

An exemplary Vibrio cholerae NanH sialidase amino acid sequence is deposited under accession number A5F7A4 and is reproduced below as SEQ ID NO: 3 (781 amino acids).

SEQ ID NO: 3
MRFKNVKKTA LMLAMFGMAT SSNAALFDYN ATGDTEFDSP
AKQGWMQDNT NNGSGVLTNA DGMPAWLVQG IGGRAQWTYS
LSTNQHAQAS SFGWRMTTEM KVLSGGMITN YYANGTQRVL
PIISLDSSGN LVVEFEGQTG RTVLATGTAA TEYHKFELVF
LPGSNPSASF YFDGKLIRDN IQPTASKQNM IVWGNGSSNT
DGVAAYRDIK FEIQGDVIFR GPDRIPSIVA SSVTPGVVTA
FAEKRVGGGD PGALSNTNDI ITRTSRDGGI TWDTELNLTE
QINVSDEFDF SDPRPIYDPS SNTVLVSYAR WPTDAAQNGD
RIKPWMPNGI FYSVYDVASG NWQAPIDVTD QVKERSFQIA
GWGGSELYRR NTSLNSQQDW QSNAKIRIVD GAANQIQVAD
GSRKYVVTLS IDESGGLVAN LNGVSAPIIL QSEHAKVHSF
HDYELQYSAL NHTTTLFVDG QQITTWAGEV SQENNIQFGN
ADAQIDGRLH VQKIVLTQQG HNLVEFDAFY LAQQTPEVEK
DLEKLGWTKI KTGNTMSLYG NASVNPGPGH GITLTRQQNI
SGSQNGRLIY PAIVLDRFFL NVMSIYSDDG GSNWQTGSTL
PIPFRWKSSS ILETLEPSEA DMVELQNGDL LLTARLDFNQ
IVNGVNYSPR QQFLSKDGGI TWSLLEANNA NVFSNISTGT
VDASITRFEQ SDGSHFLLFT NPQGNPAGTN GRQNLGLWFS
FDEGVTWKGP IQLVNGASAY SDIYQLDSEN AIVIVETDNS
NMRILRMPIT LLKQKLTLSQ N

The CBM region of SEQ ID NO: 3 is from amino acid residue 25 to 216 (sequence shown in bold)—this sequence may be SEQ ID NO: 4.

An exemplary Streptococcus pneumoniae NanA sialidase amino acid sequence has been deposited under accession number P62575 and is reproduced below as SEQ ID NO: 5 (1035 amino acids).

SEQ ID NO: 5
MSYFRNRDID IERNSMNRSV QERKCRYSIR KLSVGAVSMI
VGAVVFGTSP VLAQEGASEQ PLANETQLSG ESSTLTDTEK
SQPSSETELS GNKQEQERKD KQEEKIPRDY YARDLENVET
VIEKEDVETN ASNGQRVDLS SELDKLKKLE NATVHMEFKP
DAKAPAFYNL FSVSSATKKD EYFTMAVYNN TATLEGRGSD
GKQFYNNYND APLKVKPGQW NSVTFTVEKP TAELPKGRVR
LYVNGVLSRT SLRSGNFIKD MPDVTHVQIG ATKRANNTVW
GSNLQIRNLT VYNRALTPEE VQKRSQLFKR SDLEKKLPEG
AALTEKTDIF ESGRNGKPNK DGIKSYRIPA LLKTDKGTLI
AGADERRLHS SDWGDIGMVI RRSEDNGKTW GDRVTITNLR
DNPKASDPSI GSPVNIDMVL VQDPETKRIF SIYDMFPEGK
GIFGMSSQKE EAYKKIDGKT YQILYREGEK GAYTIRENGT
VYTPDGKATD YRVVVDPVKP AYSDKGDLYK GNQLLGNIYF
TTNKTSPFRI AKDSYLWMSY SDDDGKTWSA PQDITPMVKA
DWMKFLGVGP GTGIVLRNGP HKGRILIPVY TTNNVSHLNG
SQSSRIIYSD DHGKTWHAGE AVNDNRQVDG QKIHSSTMNN
RRAQNTESTV VQLNNGDVKL FMRGLTGDLQ VATSKDGGVT
WEKDIKRYPQ VKDVYVQMSA IHTMHEGKEY IILSNAGGPK
RENGMVHLAR VEENGELTWL KHNPIQKGEF AYNSLQELGN
GEYGILYEHT EKGQNAYTLS FRKFNWDFLS KDLISPTEAK
VKRTREMGKG VIGLEFDSEV LVNKAPTLQL ANGKTARFMT
QYDTKTLLFT VDSEDMGQKV TGLAEGAIES MHNLPVSVAG
TKLSNGMNGS EAAVHEVPEY TGPLGTSGEE PAPTVEKPEY
TGPLGTSGEE PAPTVEKPEY TGPLGTAGEE AAPTVEKPEF
TGGVNGTEPA VHEIAEYKGS DSLVTLTTKE DYTYKAPLAQ
QALPETGNKE SDLLASLGLT AFFLGLFTLG KKREQ

The CBM region of SEQ ID NO: 5 is from amino acid residue 121 to 305 (sequence shown in bold)—this sequence may be SEQ ID NO: 6.

CBMs for use in the various aspects and embodiments of this disclosure may comprise a protein or peptide having the sequence of SEQ ID NO: 3, 4, 5 or 6 or a carbohydrate binding fragment of any of these. For example, a useful molecule (namely a molecule for the uses, methods and medicaments described herein) may comprise a proteinaceous moiety encoded by the sialic acid binding domain of the nanH gene (encoding sialidase) of V. cholerae (as provided by SEQ ID NO: 3) or an equivalent or homologous gene present in another organism (for example the equivalent/homologous nanA sialidase gene of S. pneumoniae: see SEQ ID NO: 5).

A molecule for the uses, methods and medicaments of this disclosure may comprise from about residue 1, 5, 10, 15, 25 or 30 (i.e. from 1-30 or from any amino acid residue there between) to about residue 150, 175, 200, 210, 216, 220-781 (to any residue from 150 to 781 including any residue therebetween) of the V. cholerae sialidase molecule of SEQ ID NOS: 3 and 4.

For example, a use, method or medicament of this disclosure may comprise a peptide having a sequence corresponding to residue 25 to about residue 216 of SEQ ID NO: 3 above.

A carbohydrate binding fragment of SEQ ID NOS: 3 may comprise anywhere between about 5, 6, 7, 8, 9 or 10 (consecutive or contiguous) amino acids to about 191 (consecutive or contiguous) amino acids from SEQ ID NO: 3. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130 or about 135, about 140, about 150, about 155, about 160, about 165, about 170, about 180, about 185, about 186, about 187, about 188, about 189 or about 190 (consecutive or contiguous) amino acids from SEQ ID NO: 3.

A molecule for a use, method or medicament of this disclosure may comprise from about residue 1, 5, 10, 15, 25 or 30 (i.e. from 1-30 or from any amino acid residue there between) to about residue 150, 175, 200, 210, 216, 220-781 (to any residue from 150 to 781 including any residue therebetween) of the V. cholerae sialidase molecule of SEQ ID NOS: 3 and 4. For example, a sialic acid binding molecule for use may comprise a peptide having a sequence corresponding to residue 25 to about residue 216 of SEQ ID NO: 3 above.

A further suitable molecule may comprise a protein or peptide having the sequence of SEQ ID NO: 5 or 6 or a carbohydrate binding fragment thereof. For example, a useful sialic acid binding molecule may comprise a proteinaceous moiety encoded by the sialic acid binding domain of the Streptococcus pneumoniae nanA gene (encoding sialidase).

A sialic acid binding molecule for use may comprise from about residue 80, 90, 100, 110, 120, 121 to 130 (i.e. from any of about residues 80 to 130 including any residue therebetween) to about residue 250, 275, 300, 305, 310, 320-1035 (i.e. to any residue from about 250-1035 including to about any residue therebetween) of the S. pneumoniae sialidase molecule of SEQ ID NOS: 5 and 6.

For example, a sialic acid binding molecule for use may comprise a peptide having a sequence corresponding to residue 121 to about residue 305 of SEQ ID NO: 5 above.

A carbohydrate binding fragment may comprise anywhere between about 5, 6, 7, 8, 9 or 10 (consecutive or contiguous) amino acids to about 185 (consecutive or contiguous) amino acids from SEQ ID NO: 5. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, 180, 181, 182, 183 or 184 (consecutive or contiguous) amino acids from SEQ ID NO: 5.

SEQ ID NOS 3 and 4 are derived from Vibrio cholerae and SEQ ID NOS 5 and 6 are derived from Streptococcus pneumoniae. A CBM for use in the various aspects of this disclosure may comprise one, two, three, four or more CBM40s. A CBM for use may comprise one, two, three, four or more proteins comprising SEQ ID NO: 3, 4, 5 or 6 or a carbohydrate binding fragment any of these. One of skill will appreciate that useful CBM40s may comprise sequences which exhibit some degree (for example, 99%, 95%, 90%, 85%, 80%, 75%, 70%, 65% or 60%) of sequence identity or homology with the CBM40 sequences of SEQ ID NOS: 3, 4, 5 and/or 6. All such variant or divergent sequences are to be embraced within the scope of this disclosure and by the term “CBM40”. Identical and/or homologous CBM40 sequences may have carbohydrate/sialic acid binding function.

CBM47

A useful CBM47 (i.e. a CBM47 for the various methods, medicaments and uses described herein) may be obtained from microorganisms, including, for example, bacteria of the genera Acinetobacter, Bathymodiolus, Campylobacter, Planctomycetes, Streptococcus and Streptomyces. For example, useful CBM47s may be obtained or derived from, for example, Streptococcus mitis or Streptococcus pneumoniae. Further details concerning potential sources and the structure and function of the CBM47 family can be found within the Carbohydrate Active Enzymes database (freely available on the internet at: www.cazy.org/CBM47.html).

An exemplary CBM47 sequence is provided by SEQ ID NO: 7 below:

SEQ ID NO: 7
TPDKFNDGNLNIAYAKPTTQSSVDYNGDPNRAVDGNRNGNFNSGSVTHT
RADNPSWWEVDLKKMDKVGLVKIYNRTDAETQRLSNFDVILYDNNRNEV
AKKHVNNLSGESVSLDFKEKGARYIKVKLLTSGVPLSLAEVEVFRES

Accordingly, a CBM for use may comprise, consist essentially or consist of a CBM having the sequence of SEQ ID NO: 7 or a carbohydrate binding portion thereof.

A carbohydrate binding fragment of SEQ ID NO: 7 may comprise anywhere between about 5, 6, 7, 8, 9 or 10 (consecutive or contiguous) amino acids to about 144 (consecutive or contiguous) amino acids from SEQ ID NO: 7. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130 or about 135, about 140 or about 143 (consecutive or contiguous) amino acids from SEQ ID NO: 7.

CBM47 or a protein comprising, consisting essentially or consisting of SEQ ID NO: 7 may bind L-fucose, fucosyllactose, H-trisaccharide and/or LewisY antigen. Accordingly, any fragment for use may also bind L-fucose, fucosyllactose, H-trisaccharide and/or LewisY antigen.

SEQ ID NO: 7 is derived from the sequence deposited in the UniProt database under ID No: A0A1Q2T229. This sequence is reproduced as SEQ ID NO: 8 below (SEQ ID NO: 7 appears as residues 601-745—shown in bold in the sequence below):

SEQ ID NO: 8
MNKEKIKRKL ITILFVCIGM LCFGLLAGVK ADNRVQMRTT
INNESPLLLS PLYGNDNGNG LWWGNTLKGA WEAIPEDVKP
YAAIELHPAK VCKPTSCIPR DTKELREWYV KMLEEAQSLN
IPVFLVIMSA GERNTVPPEW LDEQFQKYSV LKGVLNIENY
WIYNNQLAPH SAKYLEVCAK YGAHFIWHDH EKWEWETIMN
DPTFFEASQK YHKNLVLATK NTPIRDDAGT DSIVSGFWLS
GLCDNWGSST DTWKWWEKHY TNTFETGRAR DMRSYASEPE
SMIAMEMMNV YTGGGTVYNF ECAAYTFMTN DVPTPAFTKG
IIPFFRHAIQ NPAPSKEEVV NRTKAVFWNG EGRISSLNGF
YQGLYSNDET MPLYNNGRYH ILPVIHEKID KEKISSIFPN
AKILTKNSEE LSSKVNYLNS LYPKLYEGDG YAQRVGNSWY
IYNSNANINK NQQVMLPMYT NNTKSLSLDL TPHTYAVVKE
NPNNLHILLN NYRTDKTAMW ALSGNFDASK SWKKEELELA
NWISKNYSIN PVDNDFRTTT LTLKGHTGHK PQINISGDKN
HYTYTENWDE NTHVYTITVN HNGMVEMSIN TEGTGPVSFP
TPDKFNDGNL NIAYAKPTTQ SSVDYNGDPN RAVDGNRNGN
FNSGSVTHTR ADNPSWWEVD LKKMDKVGLV KIYNRTDAET
QRLSNFDVIL YDNNRNEVAK KHVNNLSGES VSLDFKEKGA
RYIKVKLLTS GVPLSLAEVE VFRESDGKQS EEDIDKITED
KVVSTNKVAT QSSTNYEGVA ALAVDGNKDG DYGHHSVTHT
KEDSPSWWEI DLAQTEELEK LIIYNRTDAE IQRLSNFDII
IYDSNDYEVF TQHIDSLESN NLSIDLKGLK GKKVRISLRN
AGIPLSLAEV EVYTYK

SEQ ID NOS 7 and 8 are derived from Streptococcus pneumoniae.

A CBM for use in the various aspects of this disclosure may comprise one, two, three, four or more CBM47s. A CBM for use may comprise one, two, three, four or more proteins comprising SEQ ID NO: 8 or a carbohydrate binding fragment thereof.

One of skill will appreciate that useful CBM47s may comprise sequences which exhibit some degree (for example, 99%, 95%, 90%, 85%, 80%, 75%, 70%, 65% or 60%) of sequence identity or homology with the CBM47 Sequences of SEQ ID NOS: 7 and 8 All such variant or divergent sequences are to be embraced within the scope of this disclosure and by the term “CBM47”. Identical and/or homologous CBM47 sequences may have carbohydrate binding function.

CBM67

A useful CBM67 (i.e. a CBM67 for the various methods, medicaments and uses described herein) may be derived from any suitable source. For example, CBM67s for use may be obtained from microorganisms, including, for example, bacteria of the genera Bacillus, Paenibacillus, Planctomycetes and Streptomyces. For example, a useful CBM67 may be obtained or derived from, for example, Streptomyces avermitilis. Further details concerning potential sources and the structure and function of the CBM67 family can be found within the Carbohydrate Active Enzymes database (freely available on the internet at: www.cazy.org/CBM67.html).

An exemplary CBM67 sequence is provided by SEQ ID NO: 9 below:

SEQ ID NO: 9
APSLEGSSWIWFPEGEPANSAPAATRWFRRTVDLPDDITGATLAISADN
VYAVSVDGAEVARTDLEADNEGWRRPAVIDVLDHVHSGNNTLAVSASNA
SVGPAGWICVLVLTTASGEKKIFSDASWKSTDHEPADGWREPDFDDSGW
PAAKVAAAWGAGPWGRVA

Accordingly, a CBM for use may comprise, consist essentially or consist of a CBM having the sequence of SEQ ID NO: 9 or a carbohydrate binding portion thereof. A carbohydrate binding fragment of SEQ ID NO: 9 may comprise anywhere between about 5, 6, 7, 8, 9 or 10 (consecutive or contiguous) amino acids to about 164 (consecutive or contiguous) amino acids from SEQ ID NO: 9. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135 about 140, about 145, about 150, about 155, about 160 or about 163 (consecutive or contiguous) amino acids from SEQ ID NO: 9.

CBM67 or a protein comprising, consisting essentially or consisting of SEQ ID NO: 9 may bind L-rhamnose. Accordingly, any fragment for use may also bind L-rhamnose. SEQ ID NO: 9 is derived from the sequence deposited in the UniProt database under ID No: Q82PP4. This sequence is reproduced as SEQ ID NO: 10 below (SEQ ID NO: 9 appears as residues 132-296 - shown in bold in the sequence below):

SEQ ID NO: 10
MSALRVTSPS VEYVQRPLGL DAAHPRLSWP MASAAPGRRQ
SAYQVRVASS AAGLSHPDVW DSGKVVSDDS VLVPYAGPPL
KPRTRYFWSV RVWDADGGAS EWSAPSWWET GLMGASQWSA
KWISAPAPLT EAPSLEGSSW IWFPEGEPAN SAPAATRWFR
RTVDLPDDIT GATLAISADN VYAVSVDGAE VARTDLEADN
EGWRRPAVID VLDHVHSGNN TLAVSASNAS VGPAGWICVL
VLTTASGEKK IFSDASWKST DHEPADGWRE PDFDDSGWPA
AKVAAAWGAG PWGRVAPVAS AANQLRHEFR LPHKKVSRAR
LYATALGLYE AHLNGRRVGR DQLAPGWTDY RKRVQYQTYD
VTSSVRPGAN ALAAYVAPGW YAGNVGMFGP HQYGERPALL
AQLEVEYADG TSERITSGPD WRAASGPIVS ADLLSGETYD
ARKETAGWTS PGFDDRAWLA VRGADNDVPE QIVAQVDGPV
RIAKELPARK VTEPKPGVFV LDLGQNMVGS VRLRVSGDAG
TTVRLRHAEV LNPDGTIYTA NLRSAAATDT YTLKGQGEET
YEPRFTFHGF RYVEVTGFPG KPSTTSVTGR VMHTSAPFTF
EFETNVPMLN KLHSNITWGQ RGNFLSVPTD TPARDERLGW
TGDINVFAPT AAYTMESARF LTKWLVDLRD AQTSDGAFTD
VAPAVGNLGN GVAGWGDAGV TVPWALYQAY GDRQVLADAL
PSVHAWLRYL EKHSDGLLRP ADGYGDWLNV SDETPKDVIA
TAYFAHSADL AARMATELGK DAAPYTDLFT RIRKAFQTAY
VASDGKVKGD TQSAYVLTLS MNLVPDALRK AAADRLVALI
EAKDWHLSTG FLGTPRLLPV LTDTGHTDVA YRLLHQRTFP
SWGYPIDKGS TTMWERWDSI QPDGGFQTPE MNSFNHYAYG
SVGEWMYANI AGIAPGRAGY RQVVIRPRPG GEVTSARATF
ASLHGPVSTR WQQRSGGFVL TCSVPPNTTA EVWIPADHPD
RVQHTHGTFV RAEDGCAVFE VGSGSHRFTV

SEQ ID NOS 9 and 10 are derived from Streptomyces avermitilis.

A CBM for use in the various aspects of this disclosure may comprise one, two, three, four or more CBM67s.

A CBM for use may comprise one, two, three, four or more proteins comprising SEQ ID NO: 9 or a carbohydrate binding fragment thereof.

One of skill will appreciate that useful CBM67s may comprise sequences which exhibit some degree (for example 99%, 95%, 90%, 85%, 80%, 75%, 70%, 65% or 60%) of sequence identity or homology with the CBM67 Sequences of SEQ ID NOS: 9 and 10. All such variant or divergent sequences are to be embraced within the scope of this disclosure and by the term “CBM67”. Identical and/or homologous CBM67 sequences may have carbohydrate binding function.

CBM70

A useful CBM70 (i.e. a CBM70 for the various methods, medicaments and uses described herein) may be derived from any suitable source. For example, CBM70s for use may be obtained from microorganisms, including, for example, bacteria of the genera Bacillus, Paenibacillus, Planctomycetes and Streptococcus. For example, a useful CBM70s may be obtained or derived from, for example, Streptomyces pneumoniae. Further details concerning potential sources and the structure and function of the CBM70 family can be found within the Carbohydrate Active Enzymes database (freely available on the internet at: www. cazy. org/CBM 70. html)

An exemplary CBM70 sequence is provided by SEQ ID NO: 11 below:

SEQ ID NO: 11
NLVENGDFGQTEDGSSPWTGSKAQGWSAWVDQKNSADASTRVIEAKDGAI
TISSHEKLRAALHRMVPIEAKKKYKLRFKIKTDNKIGIAKVRIIEESGKD
KRLWNSATTSGTKDWQTIEADYSPTLDVDKIKLELFYETGTGTVSFKDIE
LVEVADQLS

A carbohydrate binding fragment of SEQ ID NO: 11 may comprise anywhere between about 5, 6, 7, 8, 9 or 10 (consecutive or contiguous) amino acids to about 158 (consecutive or contiguous) amino acids from SEQ ID NO: 11. Suitable fragments may comprise about 11, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155 or about 157 (consecutive or contiguous) amino acids from SEQ ID NO: 11.

CBM70 or a protein comprising, consisting essentially or consisting of SEQ ID NO: 11 may bind hyaluronan. Accordingly, any fragment for use may also bind hyaluronan.

SEQ ID NO: 11 is derived from the sequence deposited in the UniProt database under ID No: Q54873. This sequence is reproduced as SEQ ID NO: 12 below (SEQ ID NO: 11 appears as residues 54-212 - shown in bold in the sequence below):

SEQ ID NO: 12
MQTKTKKLIV SLSSLVLSGF LLNHYMTIGA EETTTNTIQQ
SQKEVQYQQR DTKNLVENGD FGQTEDGSSP WTGSKAQGWS
AWVDQKNSAD ASTRVIEAKD GAITISSHEK LRAALHRMVP
IEAKKKYKLR FKIKTDNKIG IAKVRIIEES GKDKRLWNSA
TTSGTKDWQT IEADYSPTLD VDKIKLELFY ETGTGTVSFK
DIELVEVADQ LSEDSQTDKQ LEEKIDLPIG KKHVFSLADY
TYKVENPDVA SVKNGILEPL KEGTTNVIVS KDGKEVKKIP
LKILASVKDA YTDRLDDWNG IIAGNQYYDS KNEQMAKLNQ
ELEGKVADSL SSISSQADRT YLWEKFSNYK TSANLTATYR
KLEEMAKQVT NPSSRYYQDE TVVRTVRDSM EWMHKHVYNS
EKSIVGNWWD YEIGTPRAIN NTLSLMKEYF SDEEIKKYTD
VIEKFVPDPE HFRKTTDNPF KALGGNLVDM GRVKVIAGLL
RKDDQEISST IRSIEQVFKL VDQGEGFYQD GSYIDHTNVA
YTGAYGNVLI DGLSQLLPVI QKTKNPIDKD KMQTMYHWID
KSFAPLLVNG ELMDMSRGRS ISRANSEGHV AAVEVLRGIH
RIADMSEGET KQCLQSLVKT IVQSDSYYDV FKNLKTYKDI
SLMQSLLSDA GVASVPRPSY LSAFNKMDKT AMYNAEKGFG
FGLSLFSSRT LNYEHMNKEN KRGWYTSDGM FYLYNGDLSH
YSDGYWPTVN PYKMPGTTET DAKRADSDTG KVLPSAFVGT
SKLDDANATA TMDFTNWNQT LTAHKSWFML KDKIAFLGSN
IQNTSTDTAA TTIDQRKLES GNPYKVYVND KEASLTEQEK
DYPETQSVFL ESFDSKKNIG YFFFKKSSIS MSKALQKGAW
KDINEGQSDK EVENEFLTIS QAHKQNRDSY GYMLIPNVDR
ATFNQMIKEL ESSLIENNET LQSVYDAKQG VWGIVKYDDS
VSTISNQFQV LKRGVYTIRK EGDEYKIAYY NPETQESAPD
QEVFKKLEQA AQPQVQNSKE KEKSEEEKNH SDQKNLPQTG
EGQSILASLG FLLLGAFYLF RRGKNN

SEQ ID NOS 11 and 12 are derived from Streptococcus pneumoniae.

A CBM for use in the various aspects of this disclosure may comprise one, two, three, four or more CBM70s.

A CBM for use may comprise one, two, three, four or more proteins comprising SEQ ID NO: 11 or a carbohydrate binding fragment thereof.

One of skill will appreciate that useful CBM70s may comprise sequences which exhibit some degree (for example 99%, 95%, 90%, 85%, 80%, 75%, 70%, 65% or 60%) of sequence identity or homology with the CBM70 Sequences of SEQ ID NOS: 11 and 12. All such variant or divergent sequences are to be embraced within the scope of this disclosure and by the term “CBM70”. Identical and/or homologous CBM70 sequences may have carbohydrate binding function.

The various molecules described herein (including the various carbohydrate, sialic acid and/or glycan binding proteins/molecules) which are all for the various uses, medicaments and methods described herein, may further comprise an oligomerisation domain.

Suitable oligomerisation domains may exhibit an ability to self-associate to form multimeric structures, for example trimers. An oligomerisation domain for use may comprise any molecule with oligomerisation properties or any functional fragment thereof. For example, one or more (for example, two) sialic acid/glycan binding molecules (for example, CBMs) may be bound, coupled or fused to an oligomerisation domain—the resulting sialic acid/glycan binding molecule::oligomerisation domain “fusion” may then be used (with one or more other such “fusions”) as a molecule for treating or preventing a Coronavirus infection and/or a disease or condition associated therewith.

Suitable oligomerisation domains may be derived from, for example, Pseudomonas aeruginosa pseudaminidase. An exemplary Pseudomonas aeruginosa pseudaminidase sequence amino acid sequence has been deposited under accession number PA0579 and is reproduced below as SEQ ID NO: 13 (438 amino acids).

SEQ ID NO: 13
MNTYFDIPHR LVGKALYESY YDHFGQMDIL SDGSLYLIYR
RATEHVGGSD GRVVFSKLEG GIWSAPTIVA QAGGQDFRDV
AGGTMPSGRI VAASTVYETG EVKVYVSDDS GVTWVHKFTL
ARGGADYNFA HGKSFQVGAR YVIPLYAATG VNYELKWLES
SDGGETWGEG STIYSGNTPY NETSYLPVGD GVILAVARVG
SGAGGALRQF ISLDDGGTWT DQGNVTAQNG DSTDILVAPS
LSYIYSEGGT PHVVLLYTNR TTHFCYYRTI LLAKAVAGSS
GWTERVPVYS APAASGYTSQ VVLGGRRILG NLFRETSSTT
SGAYQFEVYL GGVPDFESDW FSVSSNSLYT LSHGLQRSPR
RVVVEFARSS SPSTWNIVMP SYFNDGGHKG SGAQVEVGSL
NIRLGTGAAV WGTGYFGGID NSATTRFATG YYRVRAWI

The oligomerisation domain of SEQ ID NO: 13 is from amino acid residue 333 to 438—this sequence may be SEQ ID NO: 14 (sequence shown in bold).

Thus, an oligomerisation domain for use may comprise from about residue 250, 275, 300, 310, 320, 333, 340 to 350 (i.e. from about residue 250 to about residue 350 including from about any residue therebetween) to about residue 400, 410, 420, 430 or 438 (i.e. to about any residue from about residue 400 residue 438 including to about any residue therebetween) of the P. aeruginosa pseudaminidase trimerisation domain (PaTD) provided by SEQ ID NO: 5. For example, a useful sialic acid/glycan binding molecule may exploit an oligomerisation domain comprising residues 333 to 438 of SEQ ID NO: 13.

Sialic acid/glycan binding molecules for any of the methods, uses and/or medicaments described herein may include modified forms of any of the CBMs described herein.

The term “modified” embraces molecules which contain one or more mutations relative to a reference sequence.

A “reference sequence” may be any wild type CBM sequence. For example, a reference sequence may comprise, consist essentially of or consist of a wild type family 40 CBM sequence, e.g. the wild type CBM sequences from Vibrio cholerae NanH sialidase or Streptococcus pneumoniae NanA sialidase (it should be appreciated that similar or homologous CBMs (including CBM40s) present in other organisms are to be encompassed within the scope of the term “CBM” and/or as CBM reference sequences). A reference sequence may comprise (or consist of, or consist essentially of) any of SEQ ID NOS: 1-12 above.

Accordingly, a modified CBM sequence may be derived from a specific or particular wild type CBM sequence.

A modified CBM sequence may comprise a wild type CBM sequence which has been modified to include one or more mutations.

The one or more mutations may be functional—that is to say they may individually (and/or independently) or collectively (for example, synergistically) modulate (alter, improve or suppress/inhibit) one or more of the physiological, biological immunological and/or pharmacological properties characteristic of a wild type CBM (for example the wild type CBM from which the modified CBM is derived). In particular, the one or more mutations may:
(i) alter the immunogenicity (or antigenicity) of the CBM; and/or
(ii) alter (for example improve) the efficacy (of the CBM or of any multimeric molecule comprising a modified CBM)' and/or
(iii) they may modulate (for example improve) the thermostability of the CBM; and/or
(iv) they may modulate (for example improve) the solubility of the CBM; and/or
(v) they may modulate the binding affinity of the CBM for its target (for example a glycan, sialic acid, galactose, lactose, polygalacturonic acid, LacNAc, fucose, L-rhamnose; hyaluronan); and/or
(vi) they may modulate (for example improve) the in vivo half-life of the molecule.

A “mutation” may include any alteration to a wild-type CBM molecule. For example, the term “mutation” may embrace, for example:
(i) one or more amino acid substitution(s) (where one or more of the wild type amino acid(s) is/are swapped or changed for another (different) amino acid—the term “substitutions” would include conservative amino acid substitutions); and/or
(ii) one or more amino acid deletion(s) (where one or more of the wild type amino acid residue(s) are removed); and/or
(iii) one or more amino acid addition(s)/insertion(s) (where additional amino acid residue(s) are added to a wild type (or reference) primary sequence); and/or
(iv) one or more amino acid/sequence inversions (usually where two or more consecutive amino acids in a primary sequence are reversed; and/or
(v) one or more amino acid/sequence duplications (where an amino acid or a part of the primary amino acid sequence (for example a stretch of 5-10 amino acids) is repeated).

Accordingly, a modified CBM according to this disclosure may comprise one or more of the mutations described herein.

An exemplary wild type CBM (in other words a reference sequence from which a useful modified CBM may be derived) is the Streptococcus pneumoniae NanA sialidase, the amino acid sequence for which has been deposited under accession number P62575 and is reproduced above as SEQ ID NO: 5 (1035 amino acids). As also noted above, the CBM region of SEQ ID NO: 5 is from amino acid residue 121 to 305—this sequence is designated SEQ ID NO: 6.

Thus, this disclosure provides carbohydrate binding molecules with an affinity for sialic acid, which molecules comprise modified forms of SEQ ID NO: 6 (and/or SEQ ID NO: 5). A modified form of SEQ ID NO: 6 may comprise one or more mutated residues—the mutations being, for example, amino acid substitutions, additions/insertions, duplications, deletions and/or inversions made relative to the sequence of SEQ ID NO: 6.

An exemplary Vibrio cholerae NanH sialidase amino acid sequence is deposited under accession umber A5F7A4 and is reproduced above as SEQ ID NO: 7 (781 amino acids). The CBM region of SEQ ID NO: 7 is from amino acid residue 25 to 216—this is SEQ ID NO: 8.

Thus, this disclosure provides sialic acid/glycan binding molecules which comprise modified forms of SEQ ID NO: 8 (and/or SEQ ID NO: 7). A modified form of SEQ ID NO: 8 may comprise one or more mutated residues—the mutations being, for example, amino acid substitutions, additions/insertions, duplications, deletions and/or inversions made relative to the sequence of SEQ ID NO: 8.

Thus, a useful modified CBM (i.e. a CBM for use in the medical uses and methods described herein) may comprise one or more of the mutations described herein.

By way of non-limiting example, the following represent individual units (referred to as “HEX” units) which may be used to make modified sialic acid/glycan binding molecules for the various uses, methods and medicaments described herein (for example, for use in methods of treating or preventing Coronavirus infections).

(i) HEX1

CBMX1 (L170T V239A V246G I286A Y292E)----CBMX2 (L170T V239A V246G I286A Y292E)----TD (S342D L348D R403K)

(ii) HEX2

CBMX1 (V239A V246G I286A Y292E)----CBMX2 (V239A V246G I286A Y292E)----TD (S342D R403K)

(iii) HEX3

CBMX1 (V239A V246G I286A)----CBMX2 (V239A V246G I286A)----TD (S342D R403K)

(iv) HEX4

CBMX1 (V239A V246G)----CBMX2 (V239A V246G)----TD (S342D)

(v) HEX5

CBMX1 (V239A V246G)----CBMX2 (V239A V246G)----TD (R403K)

(vi) HEX6

CBMX1 (V239A V246G)----CBMX2 (V239A V246G)----TD (S342D R403K)

(vii) HEX17

CBMX1 (V239A V246G A162P)----CBMX2 (V239A V246G A162P)----TD (S342D R403K)

A suitable HEX unit may comprise two modified CBMs (denoted CBMX1 and CBMX2 above) with the specific mutations introduced to each CBM being identified in parenthesis. In one teaching the units CBMX1 and CBMX2 may comprise (consist of, or consist essentially of) any type of CBM (for example any member of the CBM40, any CBM32, any CBM47, any CBM67 and/or any CBM70 groups/classes). It should be noted that a “----” symbol indicates an amino acid linker (linking one modified CBM to another modified CBM or a modified CBM to an oligomerisation domain).

In each case, the oligomerisation domain (denoted “TD”) present in each HEX unit conjugates the units together as a trimer. While any given hexamer may comprise three identical copies of one of the units described above, one of skill will appreciate that further options are available. For example, a HEX unit may be made up of two CBMs, each having different mutations (the mutations being one or more selected from the options detailed herein).

It will be noted that HEX6 and HEX17 are identical except for the additional A162P mutation. This proline mutation (a substitution for the wild type alanine at residue 162) has been shown to improve thermostability (the single CBM Tm by 3-4° C.). Further information regarding the use of proline mutations may be derived from Fu 2009, ‘Increasing protein stability by improving beta-turns’ (DOI 10.1002/prot.22509) which describes the general approach. The proline mutation does not affect (increase or decrease) the predicted immunogenicity of the CBM molecule, is not located near the other mutations, the N- or C-termini or the ligand binding site. Rather unexpectedly, beyond the modest improvement in thermostability, it was noted that the A162P mutation yields a molecule exhibiting a marked improvement in in vivo experiments—in particular in comparison to those same experiments conducted using a hexameric molecule comprising other (for example, HEX6) HEX units. For example, the modified molecules (in particular, a molecule comprising a HEX17 unit) exhibit modulation over pro-inflammatory cytokines, including, for example, IL-8. Indeed, the modulatory effect (specifically an inhibitory effect) on the production of IL-8 by a molecule comprising a HEX17 unit, was improved over other tested modified molecules.

Relative to the amino acid sequences of Sp2CBMTD (aka “SpOrig”) the amino acid sequence of the HEX6 and HEX17 molecules is:

| | |
|---|---|
| SpOrig | GAMVIEKEDVETNASNGQRVDLSSELDKLKKLENATVHMEFKP |
| | DAKAPAFYNLFSVSSAT |
| HEX6 | GAMVIEKEDVETNASNGQRVDLSSELDKLKKLENATVHMEFKP |
| | DAKAPAFYNLFSVSSAT |
| HEX17 | GAMVIEKEDVETNASNGQRVDLSSELDKLKKLENATVHMEFKP |
| | DPKAPAFYNLFSVSSAT |
| SpOrig | KKDEYFTMAVYNNTATLEGRGSDGKQFYNNYNDAPLKVKPGQW |
| | NSVTFTVEKPTAELPKG |
| HEX6 | KKDEYFTMAVYNNTATLEGRGSDGKQFYNNYNDAPLKVKPGQW |
| | NSVTFTVEKPTAELPKG |
| HEX17 | KKDEYFTMAVYNNTATLEGRGSDGKQFYNNYNDAPLKVKPGQW |
| | NSVTFTVEKPTAELPKG |
| SpOrig | RVRLYVNGVLSRTSLRSGNFIKDMPDVTHVQIGATKRANNTVW |
| | GSNLQIRNLTVYNRALT |
| HEX6 | RARLYVNGGLSRTSLRSGNFIKDMPDVTHVQIGATKRANNTVW |
| | GSNLQIRNLTVYNRALT |
| HEX17 | RARLYVNGGLSRTSLRSGNFIKDMPDVTHVQIGATKRANNTVW |
| | GSNLQIRNLTVYNRALT |
| SpOrig | PEEVQKRSGGGSGVIEKEDVETNASNGQRVDLSSELDKLKKLE |
| | NATVHMEFKPDAKAPAF |
| HEX6 | PEEVQKRSGGGSGVIEKEDVETNASNGQRVDLSSELDKLKKLE |
| | NATVHMEFKPDAKAPAE |
| HEX17 | PEEVQKRSGGGSGVIEKEDVETNASNGQRVDLSSELDKLKKLE |
| | NATVHMEFKPDPKAPAF |
| SpOrig | YNLFSVSSATKKDEYFTMAVYNNTATLEGRGSDGKQFYNNYND |
| | APLKVKPGQWNSVTFTV |
| HEX6 | YNLFSVSSATKKDEYFTMAVYNNTATLEGRGSDGKQFYNNYND |
| | APLKVKPGQWNSVTFTV |
| HEX17 | YNLFSVSSATKKDEYFTMAVYNNTATLEGRGSDGKQFYNNYND |
| | APLKVKPGQWNSVTFTV |
| SpOrig | EKPTAELPKGRVRLYVNGVLSRTSLRSGNFIKDMPDVTHVQIG |
| | ATKRANNTVWGSNLQIR |
| HEX6 | EKPTAELPKGRARLYVNGGLSRTSLRSGNFIKDMPDVTHVQIG |
| | ATKRANNTVWGSNLQIR |
| HEX17 | EKPTAELPKGRARLYVNGGLSRTSLRSGNFIKDMPDVTHVQIG |
| | ATKRANNTVWGSNLQIR |
| SpOrig | NLTVYNRALTPEEVQKRSGGALGVPDFESDWFSVSSNSLYTLS |
| | HGLQRSPRRVVVEFARS |
| HEX6 | NLTVYNRALTPEEVQKRSGGSLGVPDFESDWFDVSSNSLYTLS |
| | HGLQRSPRRVVVEFARS |
| HEX17 | NLTVYNRALTPEEVQKRSGGSLGVPDFESDWFDVSSNSLYTLS |
| | HGLQRSPRRVVVEFARS |
| SpOrig | SSPSTWNIVMPSYFNDGGHKGSGAQVEVGSLNIRLGTGAAVWG |
| | TGYFGGIDNSATTRFAT |
| HEX6 | SSPSTWNIVMPSYFNDGGHKGSGAQVEVGSLNIKLGTGAAVWG |
| | TGYFGGIDNSATTRFAT |
| HEX17 | SSPSTWNIVMPSYFNDGGHKGSGAQVEVGSLNIKLGTGAAVWG |
| | TGYFGGIDNSATTRFAT |
| SpOrig | GYYRVRAWI |
| HEX6 | GYYRVRAWI |
| HEX17 | GYYRVRAWI |

[SpOrig (SEQ ID NO: 15); HEX6 (SEQ ID NO: 16) and
HEX17 (SEQ ID NO: 17)]

SpOrig may be modified to include the following mutation (R274Q). This mutation modulates 25 the binding affinity of the CBM for sialic acid.

In view of the above, the disclosure provides a modified CBM, for use in the treatment or prevention of:
(i) a Coronavirus infection and/or a disease or condition caused or contributed by a Coronavirus; or
(ii) a COVID-19 infection and/or a disease or condition caused or contributed by SARS-CoV-2.

Also disclosed is a method of treating or preventing:
(i) a Coronavirus infection and/or a disease or condition caused or contributed by a Coronavirus; or
(ii) a COVID-19 infection and/or a disease or condition caused or contributed by SARS-CoV-2;
said method comprising administering a subject in need thereof a modified CBM.

Additionally disclosed, is the use of a modified CBM in the manufacture of a medicament for the treatment or prevention of a Coronavirus infection and/or a disease or condition caused or contributed by a Coronavirus.

Additionally, the disclosure provides HEX17, for use in the treatment or prevention of:
(i) a Coronavirus infection and/or a disease or condition caused or contributed by a Coronavirus; or
(ii) a COVID-19 infection and/or a disease or condition caused or contributed by SARS-CoV-2.

Also disclosed is a method of treating or preventing:
(i) a Coronavirus infection and/or a disease or condition caused or contributed by a Coronavirus; or
(ii) a COVID-19 infection and/or a disease or condition caused or contributed by SARS-CoV-2;

said method comprising administering a subject in need thereof HEX17.

Additionally disclosed, is the use of HEX17 in the manufacture of a medicament for the treatment or prevention of a Coronavirus infection and/or a disease or condition caused or contributed by a Coronavirus.

A molecule for the uses, methods and medicaments described herein may comprise any one or more of the CBM molecules described herein. Molecules of this type may further comprise the abovementioned trimerisation domain allowing the formation of sialic acid/glycan binding molecules which are multivalent CBMs.

For example, a molecule for use may comprise a plurality or multiple (i.e. two, three, four or more) CBMs. Molecules which comprise a plurality of CBMs may be termed “multivalent glycan or sialic acid binding molecules” or “multivalent CBMs”.

Multivalent CBM molecules, may be prepared as constructs comprising multiple (identical or different) CBMs linked by amino acid/peptide linkers. Each CBM (for example, VcCBM, SpCBM or modified CBM) may be linked to another (for example, VcCBM, SpCBM or modified CBM) or to a trimerisation domain (TD) by, for example, peptides comprising 5, 10 or 15 amino acids.

By way of example any one or more of the following peptides may be used to link two or more CBMs or a CBM to a trimerisation domain, to produce a multivalent CBM:

| | | | |
|---|---|---|---|
| (i) | 5 amino acid | ALNGS | SEQ ID NO: 18 |
| | linkers: | LQALG | SEQ ID NO: 19 |
| | | GGNSG | SEQ ID NO: 20 |
| | | GGGSG | SEQ ID NO: 21 |
| | | GGALG | SEQ ID NO: 22 |
| (ii) | 10 amino acid | ALNGSGGGSG | SEQ ID NO: 23 |
| | linkers: | LQALGGGGSL | SEQ ID NO: 24 |
| (iii) | 15 amino acid | ALNGSGGGSGGGGSG | SEQ ID NO: 25 |
| | linkers: | | |

A multivalent CBM for the methods, uses and medicaments described herein may, for example, comprise two or more Family 32 CBMs, two or more Family 40 CBMs, two or more Family 47 CBMs, two or more Family 67 CBMs, two or more Family 70 CBMs. A multivalent CBM may comprise a mix of different CBM types, for example CBMs from different CBM families, or repeats of the same CBM.

Various examples of useful mono- and multivalent glycan/sialic acid binding molecules are described herein.

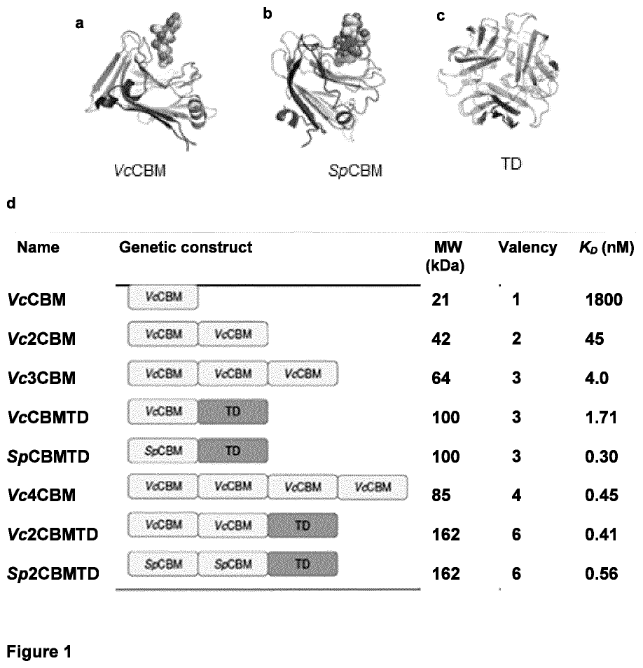

For example, the molecules presented in FIG. 1 may be for use in the methods, uses and medicaments of this disclosure. A suitable molecule may comprise (consist essentially of, or consist of) one or more (for example, two, three, four or more) VcCBMs (that is a CBM40 derived from Vibrio cholerae). The CBM (for example the VcCBM) may be fused, bound or conjugated to an oligomerisation domain (such as a PaTD or oligomerisation fragment thereof). A glycan/sialic acid binding molecule may comprise, consist or consist essentially of two fused (or bound) CBMs which are in turn fused to an oligomerisation domain (see, for example, molecule Vc2CBMTD shown in FIG. 1).

Every one of the disclosed molecules alone or in combination with another disclosed molecule, may be for use in treating or preventing a Coronavirus infection or a disease or conditions associated therewith a method of treating a Coronavirus infection or a disease or conditions associated therewith or for use in the manufacture of a medicament for treating or preventing a Coronavirus infection or a disease or conditions associated therewith.

Any of the disclosed molecules (for the uses, medicaments and methods described herein)—especially the modified CBM molecules, may be generated using PCR-based cloning techniques and a suitable method for the generation of multivalent molecules of this type is described in, for example, Connaris et al, 2009 (Enhancing the Receptor Affinity of the Sialic Acid-Binding Domain of Vibrio cholerae Sialidase through Multivalency; J. Biol. Chem; Vol. 284(11); pp 7339-7351). For example, multivalent CBM molecules, including the likes of HEX17, Vc2CBM, Vc4CBM and Sp2CBM may be prepared as constructs comprising multiple CBMs linked by amino acid/peptide linkers—such as those described above.

In the context of this disclosure, molecules (carbohydrate binding molecules, sialic acid binding molecules, glycan binding proteins/molecules and/or CBMs) for use in treating or preventing Coronavirus infections, may comprise one or more CBMs selected from the group consisting of:
(i) one or more (for example, 2, 3, 4 or more) Family 32 CBMs;
(ii) one or more (for example, 2, 3, 4 or more) Family 40 CBMs;
(iii) one or more (for example, 2, 3, 4 or more) Family 47 CBMs;
(iv) one or more (for example, 2, 3, 4 or more) Family 67 CBMs;
(v) one or more (for example, 2, 3, 4 or more) Family 70 CBMs; and
(vi) a modified CBM (as described herein).

Additionally, molecules (carbohydrate binding molecules, sialic acid binding molecules, glycan binding molecules/proteins and/or CBMs) for use in treating or preventing Coronavirus infections, may comprise a CBM selected from the group consisting of:
(i) a Clostridium perfringens CBM32 (CpCBM32);
(ii) a Streptococcus pneumoniae CBM40 (SpCBM40);
(iii) a Vibrio cholerae CBM40 (VcCBM40);
(iv) a Streptococcus pneumoniae CBM47(SpCBM47);
(v) a Streptomyces avermitilis CBM67 (SaCBM67);
(vi) a Streptococcus pneumoniae CBM70 (SpCBM70);
(vii) a Vibrio cholerae NanH sialidase CBM;
(vii) a Vibrio cholerae NanH sialidase CBM sialic acid binding fragment thereof.
(xi) a Streptococcus pneumoniae nanA sialidase CBM; and
(x) a Streptococcus pneumoniae nanA sialidase CBM sialic acid binding fragment thereof.

A multivalent CBM for the various uses, methods and medicaments described herein may comprise:
(i) a VcCBM; or
(ii) two (or more) VcCBM(s); or
(iii) three or four (or more) VcCBM(s); or
(iv) a SpCBM; or
(v) two (or more) SpCBM(s); or
(vi) three or four (or more) SpCBM(s); or
(vii) a CpCBM; or
(viii0 two (or more) CpCBM(s); or
(viii) three or four (or more) CpCBM(s); or
(ix) a SaCBM; or
(x) two (or more) SaCBM(s); or
(xi) three or four (or more) SaCBM(s).

A multivalent CBM for a use, method or medicament of this disclosure may comprise a mixture of different CBMs, for example, one or more CBM32s with one more other CBMs selected from the group consisting of:
(i) a Family 40 CBM
(ii) a Family 47 CBM
(iii) a Family 67 CBM
(iv) a Family 70 CBM

For example, a method, use or medicament described herein may exploit the combination of a CBM32 with a Family 40 CBM (a CBM40).

Alternatively, a multivalent CBM for the uses, methods and medicaments described herein may comprise a mixture of different CBMs, for example one or more CBM40s with one more other CBMs selected from the group consisting of:
(i) a Family 32 CBM
(ii) a Family 47 CBM
(iii) a Family 67 CBM
(iv) a Family 70 CBM

For example, a method, use or medicament described herein may exploit the combination of a CBM40 with a Family 32 CBM (a CBM32).

A multivalent CBM for the uses, methods and medicaments described herein may comprise a mixture of different CBMs, for example one or more CBM47s with one more other CBMs selected from the group consisting of:
(i) a Family 32 CBM
(ii) a Family 40 CBM
(iii) a Family 67 CBM
(iv) a Family 70 CBM

A multivalent CBM for the uses, methods and medicaments described herein may comprise a mixture of different CBMs, for example one or more CBM67s with one more other CBMs selected from the group consisting of:
(i) a Family 32 CBM
(ii) a Family 40 CBM
(iii) a Family 47 CBM
(iv) a Family 70 CBM

A multivalent CBM for the uses, methods and medicaments described herein may comprise a mixture of different CBMs, for example one or more CBM70s with one more other CBMs selected from the group consisting of:
(i) a Family 32 CBM
(ii) a Family 40 CBM
(iii) a Family 47 CBM
(iv) a Family 67 CBM

Multivalent CBMs for the various uses, methods and medicaments disclosed herein may include, for example, molecules selected from the group consisting of:
(i) Cp2CBM32TD (comprising, consisting essentially of or consisting of: 2 CBMs (CBM32s) from Clostridium perfringens fused to a trimerisation domain);
(ii) Sp2CBM40TD (comprising, consisting essentially of or consisting of: 2 CBMs (CBM40s) from Streptococcus pneumoniae fused to a trimerisation domain;
(iii) Vc2CBM40TD (comprising, consisting essentially of or consisting of: 2 CBMs (CBM40s) from Vibrio cholerae fused to a trimerisation domain; and
(iv) Vc4CBM (comprising, consisting essentially of or consisting of: 4 CBMs (CBM40s) from Vibrio cholerae)
(v) Sp2CBM47TD comprising, consisting essentially of or consisting of: 2 CBMs (CBM47s) from Streptococcus pneumoniae fused to a trimerisation domain);
(vi) Sa2CBM67TD comprising, consisting essentially of or consisting of: 2 CBMs (CBM67s) from Streptococcus avermitilis fused to a trimerisation domain);
(vii) Sp2CBM70TD comprising, consisting essentially of or consisting of: 2 CBMs (CBM70s) from Streptococcus pneumoniae fused to a trimerisation domain).

It should be noted that this disclosure provides uses (compositions, methods and medicaments) which comprise not only any one or more of the CBMs disclosed herein, in isolated form (that is uses in which at least the CBM component (of the therapeutic molecule) comprises or consists essentially of one or more of the CBM sequence(s) described herein and/or functional fragments thereof), but also uses in which the CBM component of the therapeutic molecule is comprised within a larger molecule. By way of example, the various CBMs described herein may be provided and/or used in the form of large molecules comprising a CBM component. The CBM component (e.g. the sialic acid binding molecule) may itself comprise (consist of or consist essentially of), for example, any one of the CBMs described herein (including, for example CBM32, CBM40, CBM47, CBM67 and CBM70). By way of (non-limiting) example, molecules (e.g. the CBMs and/or glycan/sialic acid binding molecules) of this disclosure may not only exhibit an ability to bind a glycan (or a component thereof) or sialic acid, but may also have one or more other functions. For example, the molecules may have enzymatic activity. For example, a useful molecule may comprise a CBM (as described herein) and exhibit some sialidase activity.

A useful molecule may be a fusion protein comprising an enzymatic portion and a glycan/sialic acid binding portion—wherein the glycan/sialic acid binding portion comprises a CBM as described herein. In such cases, the enzymatic portion may be fused to the glycan/sialic acid binding portion. As stated, the enzymatic portion of any useful fusion protein may comprise (or have, or exhibit) sialidase activity.

In one embodiment, the sialic acid binding molecule, glycan binding molecule, or CBM for the various uses described herein, may not be provided as part of, or comprised within, a molecule (for example a fusion protein) with enzymatic (for example sialidase) activity. Additionally or alternatively, the sialic acid/glycan binding molecule may not (i) bind heparin or heparin sulfate and/or (ii) comprise the GAG-binding domain of a protein that binds heparin or heparin sulfate moieties. A construct comprising the sialic acid/glycan binding molecule, glycan binding molecule, or CBM, may not show or exhibit enzymatic (for example sialidase) activity.

This disclosure also provides compositions, in particular pharmaceutical compositions which may be exploited in the described uses, methods and medicaments.

As such, any of the useful molecule(s) (for example, the CBMs (modified, multivalent or otherwise) or glycan binding molecules) described herein may be formulated for subsequent use. For convenience, it should be noted that the term “CBM” embraces, all monovalent, multivalent and modified CBM molecules described herein.

For example, a sialic acid binding molecule, a glycan binding molecule or CBM may be formulated as a therapeutic or pharmaceutical composition. The various compositions may comprise one or more of the sialic acid binding molecule(s)/glycan binding molecule(s)/CBM(s) as described herein and one or more pharmaceutically acceptable excipients. For example, the pharmaceutical preparations comprising the molecules described herein may be mixed with stabilisers, wetting agents, emulsifiers, salts (for use in influencing osmotic pressure), buffers and/or other substances that do not react deleteriously with the active compounds.

Any given therapeutic use or method of treatment may require the administration (together, concurrently or separately) of one or more of these compositions, which compositions may comprise one or more different CBMs.

Pharmaceutical compositions according to the present disclosure may be prepared for oral, mucosal, intranasal or parenteral (intravenous) administration. Those formulations for mucosal or intranasal administration may be prepared conventionally, comprising substances that are customarily used in pharmaceuticals and as described in, for example, Remington's The Sciences and Practice of Pharmacy, 22nd Edition (Pharmaceutical Press 2012) and/or Handbook of Pharmaceutical Excipients, 7th edition (compiled by Rowe et al, Pharmaceutical Press, 2012)—the entire content of all of these documents and references being incorporated by reference.

Liquid dosage forms for oral and/or intranasal administration may include emulsions, solutions, suspensions, syrups, and elixirs. In addition to the compound or composition, the liquid dosage forms may contain inert diluents commonly used in the art, such as water or other solvents, solubilising agents and emulsifiers.

Any suitable amount of a sialic acid binding molecule, glycan binding molecule or CBM may be used. For example, whether a composition comprising a sialic acid binding molecule, glycan binding molecule or CBM is to be administered intravenously or mucosally (for example, intranasally) the dose of sialic acid binding molecule/glycan binding molecule/CBM may comprise anywhere between about 0.1 μg and about 6000 μg. For example, a dose of about (for example +/−0.5 μg) 0.1 μg, 0.5 μg, 1 μg, 5 μg, 10 μg, 11 μg, 12 μg, 13 μg, 14 μg, 15 μg, 20 μg, 30 μg, 40 μg, 50 μg, 100 μg, 200 μg, 300 μg, 400 μg, 500 μg, 600 μg, 700 μg, 800 μg, 900 μg, 950 μg 1000μg, 1500μg, 2000μg, 2500μg, 3000μg, 3500μg, 4000μg, 4500μg, 5000μg, 5500μg, or 6000μg of the sialic acid binding molecule/glycan binding molecule/CBM may be used. These amounts may be provided in any suitable volume of excipient, diluent or buffer. For example, the amount of sialic acid binding molecule/glycan binding molecule/CBM may be provided in anywhere between about 1 μl to about 0.5 ml of excipient, diluent or buffer. For example, the required amount of sialic acid binding molecule/glycan binding molecule or CBM may be combined (or formulated) with about 5 μl, 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 140 μl 200 μl, 280 μl, 300 μl, 400 μl, 500 μl, 560 μl, 600 μl, 700 μl, 800 μl, 900 μl, or 1 ml, Concentrations of 0.1-15 mg (for example 0.5 mg, 1 mg, 2 mg, 3 mg, 4 mg, 5 mg, 6 mg, 7 mg, 8 mg, 9 mg, 10 mg, 11 mg, 12 mg, 13 mg or 14 mg) (sialic acid/glycan binding protein) per ml (excipient, diluent or buffer) may be most useful. A concentration of 10 mg/ml (excipient, diluent or buffer) may be very useful.

A composition of this disclosure (for example, a composition comprising any of the sialic acid binding molecules, glycan binding molecules or CBMs disclosed herein) may be administered (prophylactically) to a subject at regular and/or predetermined times. For example, a composition described herein may be administered at regular and/or predetermined times both before, during and after the subject enters or encounters a scenario during which they might be vulnerable and/or susceptible to a Coronavirus infection.

A composition of this disclosure may be administered every day and/or every few days.

A composition of this disclosure may be administered multiple times throughout any given day.

A composition described herein may be administered over a period of weeks or months or years. The precise administration regimen will depend on the subject, the health of that subject and the period of time that subject is deemed to be at risk of or vulnerable to, a Coronavirus infection.

The present disclosure will now be described with reference to the following figures which show:

FIG. 1: Building blocks of the multivalent CBM forms and their affinities for sialic acid. a, VcCBM, residues 25-216 of the V. cholerae sialidase (PDB:1w0p) with α-2,3-sialyllactose drawn as spheres. b, SpCBM, residues 121-305 of S. pneumoniae NanA sialidase with α-2,3-sialyllactose (PDB:4c1w). c, TD, the trimerisation domain, residues 333-438, of the P. aeruginosa pseudaminidase (PDB:2w38) in rainbow colours; the other two monomers in single colours. d, Multivalent forms: their molecular weights, valencies and binding affinities for α2,3-sialyllactose as determined by surface plasmon resonance (SPR) at 25° C. (KD values for VcCBM, Vc2CBM and Vc3CBM had been reported previously (Connaris et al, 2009)). Tandem repeat CBMs, and oligomeric CBMs fused to TD are linked by a 5-amino linker (details in Connaris, H. et al., (2014). PNAS 111:6401-6406).

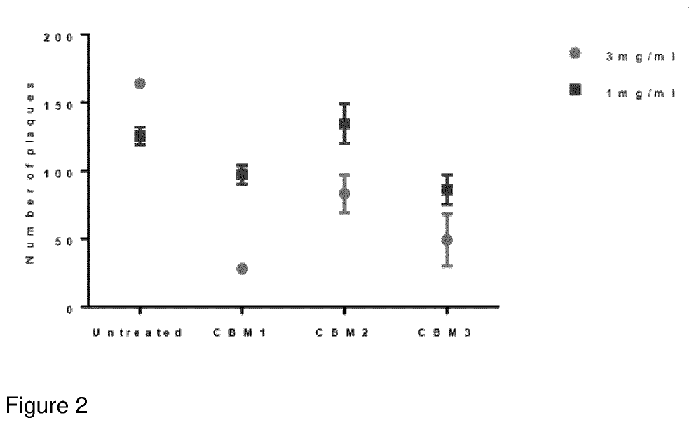

FIG. 2: Graph showing the results of the condition 1 assay. There is an observable anti-viral effect with all CBM compounds especially when tested at 3 mg/mL.

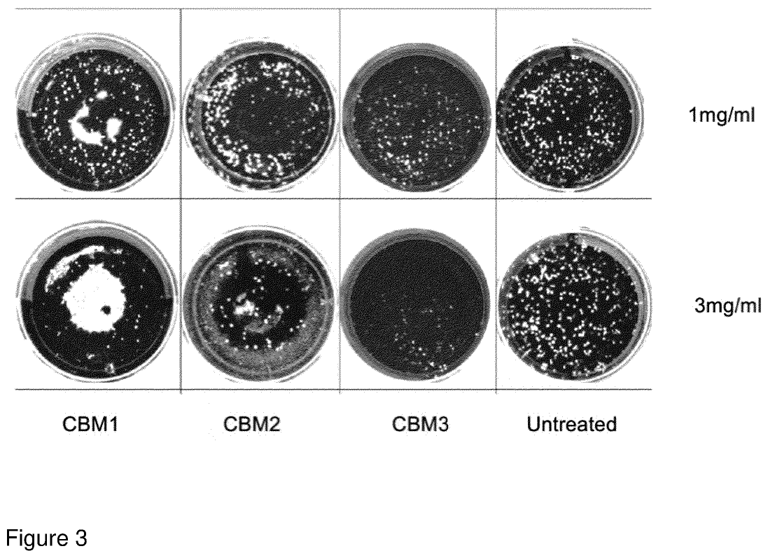

FIG. 3: Plaque assay showing the results of the condition 1 assay; again, there is an anti-viral effect shown for all 3 CBMs especially when used at the higher concentration.

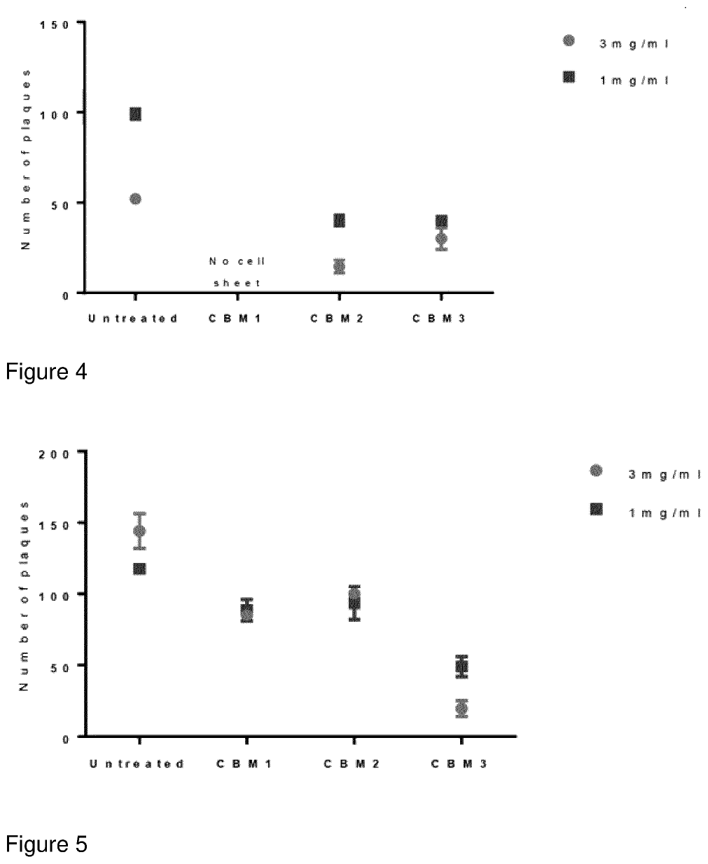

FIG. 4: Graph showing the results of the condition 2 assay. In this case, cells were exposed to CBM before SARS-CoV-2 infection; thus, the condition 2 assay represents a prophylaxis model. An anti-viral effect is shown for at least CBM2 & CBM3

FIG. 5: Graph showing the results of the condition 3 assay. In this case, infected cells (with SARS-CoV-2) were treated with the various CBMs. An anti-viral effect is shown for all 3 CBMs—particularly CBM3.

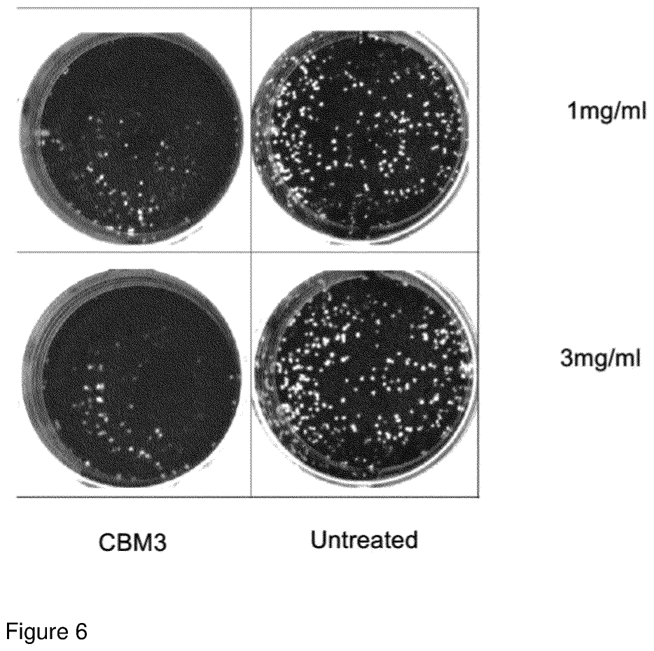

FIG. 6: Plaque assay showing the results of the condition 3 assay. Again, this shows the anti-viral effect of all 3 CBMs—particularly CBM3.

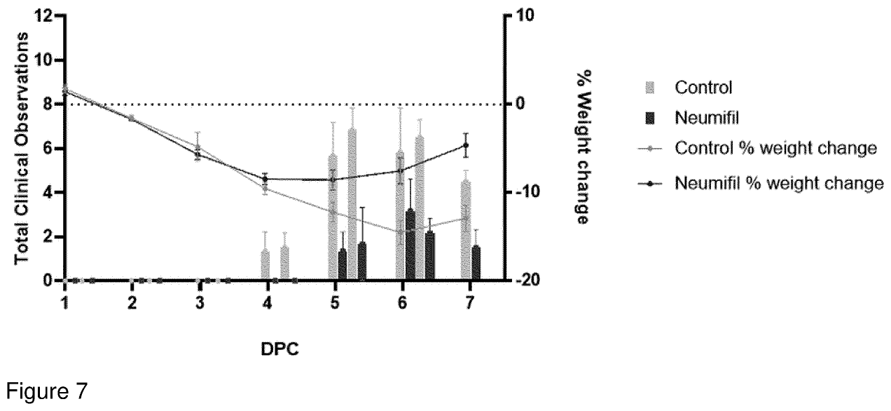

FIG. 7: the mean and SEM of the total clinical observations for group 2 (Control) and 3 (Neumifil) from 0 DPC until day of cull (7 DPC). FIG. 7 also shows the mean (with SEM) percentage weight change for the same period (right y-axis).

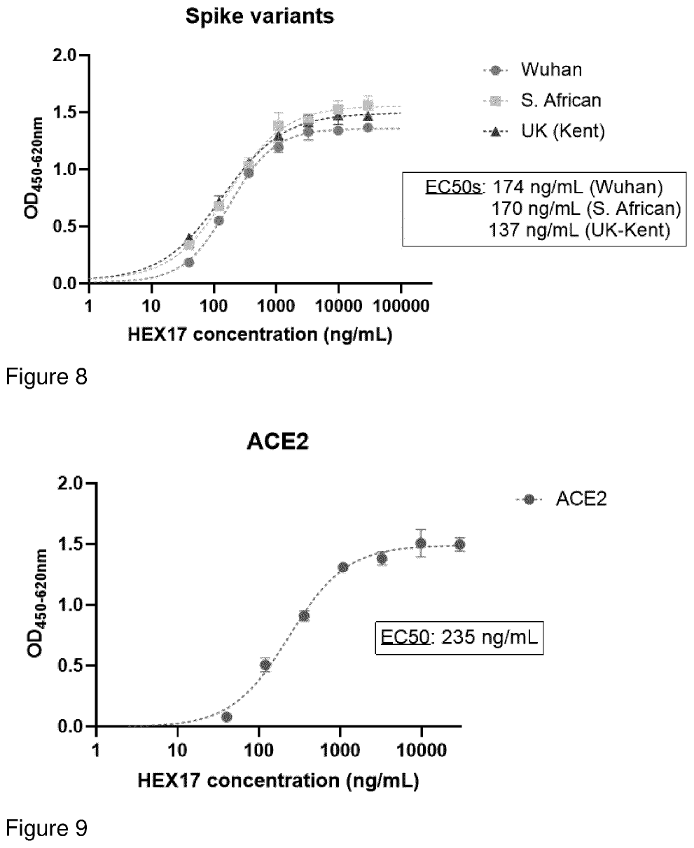

FIG. 8: Detection of Neumifil (HEX17) binding to SARS-CoV-2 Spike S1 variants. The dotted lines represent 4PL curve fits of the data. Inset: EC50 values for each variant.

FIG. 9: Detection of Neumifil (HEX17) binding to recombinant human ACE2. The dotted line represents a 4PL curve fitting of the data. Inset: EC50 value.

### METHODS

Plaque Reduction Assay

Thaw vial of titrated SARS CoV-2 on ice.

Prepare 2 working stocks of SARS-CoV-2, diluting the virus in serum free (SF) DMEM to 500 pfu/mL (working stock for condition 1) and 250 pfu/mL (working stock for conditions 2 and 3). Store the prepared stocks on ice.

For test agent preparation, thaw an aliquot of each CBM (100 μL per vial at 10 mg/mL) on ice and transfer contents into a new sterile 1.5 mL Eppendorf tube an centrifuge at 13,000 rpm for 5 min to pellet any precipitates that may have formed. Transfer the supernatant into new sterile 1.5 mL Eppendorf tubes.

From the CBM supernatants, prepare 2 working stock concentrations (3 mg/mL and 1 mg/mL, from a master stock of 10 mg/mL) of the 3 CBMs to be tested, diluting the CBMs in a 50:50 mix of serum-free DMEM:PBS (see Table 1)

The final concentration of CBMs will vary between conditions. Condition 1: final CBM concentrations 1.5 mg/mL and 0.5 mg/mL, and for conditions 2 and 3, final CBM concentrations 3 mg/mL and 1 mg/mL.

TABLE 1

| | | | | Diluent* | Vials |
|---|---|---|---|---|---|
| | [Working] | Final | CBM | volume | required/ |
| Condition | (mg/mL) | volume (μl) | volume (μL) | (μL) | CBM |
| 1 | 3 | 400 | 120 | 280 | |
| | 1 | 400 | 40 | 360 | |
| | | Total CBM | 160 | | 2 |
| | | needed (μL) | | | |
| | | | | Diluent* | Vials |
| | [Working] | Final | CBM | volume | required/ |
| | (mg/mL) | volume (μl) | volume (μL) | (μL) | CBM |
| 2 | 3 | 500 | 150 | 350 | |
| | 1 | 500 | 50 | 450 | |
| | | Total CBM | 200 | | 2 |
| | | needed (μL) | | | |
| 3 | 3 | 500 | 150 | 350 | |
| | 1 | 500 | 50 | 450 | |
| | | Total CBM | 200 | | 2 |
| | | needed (μL) | | | |

*50-50 mix of serum free DMEM:PBS

Condition 1: virus and CBM mixed prior to adding to cells
Mix equal volumes of SARS CoV-2 and CBM and incubate on ice for 1 hour. For the positive control mix SARS-CoV-2 with SF diluent (50-50 mix of SF DMEM and PBS) in place of the CBM. For the negative control mix CBM 1 at 3 mg/mL with SF DMEM in place of SARS-CoV-2.
   Tube 1: 300 μL SARS-CoV-2 at 500 pfu/mL plus 300 μL CBM1 at 3 mg/mL
   Tube 2: 300 μL SARS-CoV-2 at 500 pfu/mL plus 300 μL CBM2 at 3 mg/mL
   Tube 3: 300 μL SARS-CoV-2 at 500 pfu/mL plus 300 μL CBM3 at 3 mg/mL
   Tube 4: 300 μL SARS-CoV-2 at 500 pfu/mL plus 300 μL CBM1 at 1 mg/mL
   Tube 5: 300 μL SARS-CoV-2 at 500 pfu/mL plus 300 μL CBM1 at 1 mg/mL
   Tube 6: 300 μL SARS-CoV-2 at 500 pfu/mL plus 300 μL CBM1 at 1 mg/mL
   Tube NC: negative control: 300 μL SF DMEM plus 300 μL CBM diluent
   Tube PC: positive control, 300 μL SARS-CoV-2 at 500 pfu/mL plus 300 μL CBM diluent
After 1 hr, remove serum free media from the cells and wash with sterile PBS (<0.5 mL/well)
After the PBS wash, add 200 μL of the prepared inoculums to the appropriate wells, with tubes 1-3 (and controls) on plate 1 and tubes 4-6 (and controls) on plate 2.
Place plates in a flat-bottomed, sealable container and transfer to incubator. Incubate
at 37° C. 5% CO₂ for 1 hr.
After 1 hr, remove the inoculum and add 1 mL overlay to each well.
Transfer plates to a flat-bottomed sealable container
Incubate for 5 days at 37° C. 5% CO₂.

Condition 2: cells exposed to CBM before virus infection
Remove serum free DMEM and wash with Sterile PBS (<0.5 mL/well).
After the PBS wash, add 200 μL CBM, with CBMs at 3 mg/mL on plate 3 and 1 mg/mL on plate 4. Incubate at 37° C. 5% CO₂ for 1 hr.
After 1 hr, remove CBM and wash with sterile PBS (<0.5 mL/well).
After the PBS wash, add 200μL SARS-CoV-2 diluted to 250 pfu/mL to each well except the negative control wells. To the negative control wells, add 200 μL SF DMEM.
Place plates in a flat-bottomed, sealable container and transfer to incubator. Incubate at 37° C. 5% CO₂ for 1 hr.
After 1 hr, remove the inoculum and add 1 mL overlay to each well.
Transfer plates to a flat-bottomed sealable container
Incubate for 5 days at 37° C. 5% CO₂.

Condition 3: cells infected with SARS-CoV-2 and then treated with CBM.
Remove serum free DMEM and wash with sterile PBS (0.5 mL/well)
After the PBS wash, add 200 μL SARS-CoV-2 diluted to 250 pfu/mL to each well except the negative control. To the negative control, add 200 μL SF DMEM.
Place plates in a flat-bottomed, sealable container and transfer to incubator. Incubate at 37° C. 5% CO₂ for 1 hr.
After 1 hr, remove the inoculum and add 200 μL CBM to each well, except the negative control (CBMs at 3 mg/mL on plate 5 and 1 mg/mL on plate 6). To the negative control, add 200 μL CBM diluent (SF DMEM-PBS).
Place plates in a flat-bottomed sealable container and transfer to incubator. Incubate at 37° C. 5% CO₂ for 1 hr.
After 1 hr, remove the CBMs and add 1 mL overlay to each well.
Transfer plates to a flat-bottomed sealable container
Incubate for 4 days at 37° C. 5% CO2.

Specific details:

CBMs:
CBM1: Vc2CBM40TD (trimeric (hexavalent) form based on V. cholerae CBM40)
CBM2: Neumifil (HEX17: trimeric (hexavalent) form based on S. pneumoniae CBM40)
CBM3: Cp2CBM32TD (trimeric (hexavalentform based on C. perfringens CBM32) CBMs added at two concentrations (1 mg/mL & 3 mg/mL), and (as stated) tested in three conditions:
Condition 1: SARS-CoV-2 and CBM mixed prior to adding to cells
Condition 2: Cells exposed to CBM before SARS-CoV-2 infection (prophylaxis model)
Condition 3: Cells infected with SARS-CoV-2 and then CBM added (treatment model)

For making up of CBM:
3 mg/mL: 450 μL stock +1050 μL DMEM:PBS.
1 mg/mL: 150 μL stock +1350 μL DMEM:PBS

SARS-CoV-2
Vial labelled: SARS CoV2, England 2, P2 HCM/V/52, 05.03.20
Stock at 2.4×10⁵ pfu/mL
   →100 μL stock +900 μL media=2.4×10⁴ pfu/mL
   →200 μL above +1800 μL media=2.4×10³pfu/mL
   →1.5 mL above +6 mL media =480 pfu/mL ←Used for studies

VeroE6 cells
P15, split 1:2 25/3/20 (obtained from PHE HCM group)

Reagent Details
DMEM Sigma D5796, Lot RNBH6732, Exp Jun. 2020
PBS Gibco 10010-023, Lot 2098597, Exp 30 Jun. 2021
Overlay (aliquots made, each sufficient for 2 μlates):
   7.5 mL 4% CMC PHE media, MR/19/1044, Prod date 4 Dec. 19
   7.5 mL 2% CMC PHE media, MR/19/1043, Prod date 4 Dec. 19
   15 mL 2xMEM Gibco 21935-028, Lot 2150451, Exp 31 Dec. 2020
   800 μL FCS Gibco 10100-139, Lot 216386RP, Exp May 2024
   400 μL anti-anti Sigma A5955, Lot 035M4800V

RESULTS

Condition 1: Virus and CBM mixed prior to adding to cells

In this phase of testing, SARS-CoV-2 virus and the CBM compounds were incubated for 1 hour prior to addition to cells. Plate 1 were compounds tested at a working concentration of 3 mg/mL and plate 2 at 1 mg/mL.

Negative control wells were all intact and the positive control gave counts between 119-164 plaques per well. The results are shown in FIGS. 2 (graph) and 3 (plaque assay). The results show that there is an observable anti-viral effect with all CBM compounds.

3.2. Condition 2: Cells exposed to CBM before virus infection

In this phase of testing, VeroE6 cells were exposed to CBM compounds prior to addition of SARS-CoV-2 virus. Plate 3 were compounds tested at a working concentration of 3 mg/mL and plate 4 at 1 mg/mL. Plaques were counted after staining.

As shown in FIG. 4, there was an observable anti-viral effect with the CBM2 and CBM3 compounds when tested at a working concentration of 1 mg/mL.

3.3. Condition 3: Cells infected with SARS-CoV-2 and then treated with CBM

In this phase of testing, VeroE6 cells were infected with SARS-CoV-2 virus before addition of CBM compounds. Plate 5 were compounds tested at a working concentration of 3 mg/mL and plate 6 at 1 mg/mL. Plaques were counted after staining. Negative control wells were all intact and the positive control gave counts between 117-132 plaques per well.

When plotted on a graph (see FIG. 5), all 3 CBM compounds are shown to have an anti-viral effect, particularly CBM3 which showed the greatest reduction. Results from both working concentrations of CBM1 and CBM2 were similar. The results of the plaque assay are also shown in FIG. 6.

Conclusion

All CBMs showed some anti-viral activity against SARS-CoV-2 virus in vitro CBM2 (Neumifil: Hex17) and CBM3 (CBM32-based) are very promising. The results demonstrated here provide the basis for urgent in vivo studies in a suitable animal model.

### EXAMPLE 2

### Testing CBM vs 0C43

OC43 is a human coronavirus OC43-(HCoV-OC43); it is a Betacoronavirus and is associated with occurrences of a ‘common cold’ type illness. Its S protein binds to sugar-based receptor-determinants, specifically to 9-O-acetylated sialic acids (9-O-Ac-Sias) attached as terminal residues to glycan chains on glycoproteins and lipids.

A series of experiments were completed to determine whether or not CBMs could be used to treat or prevent OC43 infections.

In each experiment the following CBMs were used:
1) Vc2CBM40TD (VC2)
2) Cp2CBM32TD (CBM32)
3) HEX17

Each CBM was used at 1 mg/ml and at 3 mg/ml.

Condition 1: Prophylactic CBM treatment

Cells were treated with CBMs for 1 hour before incubation with hCoV-OC43 for 1 hour. The assay showed that treatment with any of the CBMs tested was able to prevent a subsequent hCoV-OC43 infection as compared to cells infected but not pre-treated with any CBM. The effect was most pronounced with VC2 (at 3 mg/ml and at 1 mg/ml), HEX17 (at 1 mg/ml) and CBM32 (at 3 mg/ml and 1 mg/ml).

Condition 2: Simultaneous CBM treatment

Cells treated with CBMs and hCoV-OC43 for 1 hour simultaneously. The assay showed that simultaneous treatment with any of the CBMs and at any concentration (1 mg/ml or 3 mg/ml) was able to reduce hCoV-OC43 infection as compared to cells infected but not treated with any CBM.

### EXAMPLE 3

Animal (Hamster) Studies Testing Neumifil Against SARS-CoV-2

Protocol/Study Design

TABLE 2
detail study protocol

| | | Action/Procedure/Sample | |
|---|---|---|---|
| | | (to include all animals | |
| | | related activity other than | |
| | | routine husbandry i.e. | |
| Study Day | | Licenced procedure, clinical | |
| (DPC- | | scoring, weight/temperature | |
| days post | Group/Number of | measurement, | |
| challenge) | animals | scheduled kill/necropsy) | Number of samples |
| −5 (−12 DPC) | 27 Hamsters | Delivery of animals | n/a |
| 0 (−7 DPC) | 27 Hamsters, | Chip all 27 animals (4 | n/a |
| | groups 1-5 | groups × 6 animals, and 1 | |
| | | group × 3 animals) | |
| | | assigned as following: | |
| | | Group 1 (Mock | |
| | | Challenge/Sentinel-PBS 5% | |
| | | Glycerol), n = 3 (3M) | |
| | | Group 2 (Challenge Control- | |
| | | PBS 5% Glycerol), n = 6 | |
| | | (3M, 3F) | |
| | | Group 3 (Challenge- | |
| | | Neumifil), n = 6 (3M, 3F) | |
| | | Group 4 (Challenge Control- | |
| | | PBS 5% Glycerol), n = 6 | |
| | | (3M, 3F) | |
| | | Group 5 (Challenge- | |
| | | Neumifil), n = 6 (3M, 3F) | |
| | | Give 1st Dose to groups 1-5: | |
| | | (1) PBS 5% Glycerol-100 ul | |
| | | intranasal (50 ul per nare) | |
| | | (2) PBS 5% Glycerol-100 ul | |
| | | intranasal (50 ul per nare) | |
| | | (3) Neumifil, 5000 ug/kg- | |
| | | 100 ul intranasal (50 ul per | |
| | | nare) | |
| | | (4) PBS 5% Glycerol-100 ul | |
| | | intranasal (50 ul per nare) | |
| | | (5) Neumifil, 5000 ug/kg- | |
| | | 100 ul intranasal (50 ul per | |
| | | nare) | |
| | | Temperature check-daily | |
| | | Clinical parameters measured | |
| | | (scored), then checked daily | |
| | | until | |
| | | Challenge, then twice-daily | |
| | | thereafter) | |
| 4 (−3 DPC) | 27 Hamsters, | (Clinical observations (scored) | n/a |
| | groups 1-5 | and temperature checked | |
| | | daily) | |
| | | Groups 1-5: Give 2nd Dose- | |
| | | same as previous | |
| 6 (−1 DPC) | 27 Hamsters, | (Clinical observations (scored) | 6 × Throat swabs-‘media tube’ |
| | groups 1-5 | and temperature checked | |
| | | daily) | |
| | | Groups 1-5: Give 3rd Dose- | |
| | | same as previous | |
| | | Also group 2: Throat swab | |
| 7 (0 DPC) | 24 Hamsters, | Challenge with SARS-CoV2 | n/a |
| | groups 2-5 | virus −5E+04 PFU groups 2&3 | |
| | | 1E+02 | |
| | | Groups 4&5 | |
| | | (No challenge for group 1) | |
| | | (Clinical observations (scored) | |
| | | and temperature checked | |
| | | twice daily, from | |
| | | hereafter) | |
| 9 (2 DPC) | 6 Hamsters, | (Clinical observations (scored) | 6 × Throat swabs-‘media tube’ |
| | Group 2 | and temperature checked | |
| | | twice daily) | |
| | | Group 2: Throat swab | |
| 10 (3 DPC) | 12 Hamsters, | (Clinical observations (scored) | 12 × 400 ul Nasal Wash −1.5 ml sarstedt |
| | groups 4 and 5 | and temperature checked | 12 × Throat Swab-‘media tube’ |
| | | twice daily) | 12 × Lung in 1.5 ml sarstedt |
| | | Terminate animals from | 12 × Lung in RNAlater tube |
| | | groups 4 and 5 (12 animals) | Histology: Nasal cavity*, |
| | | Collect, per animal: | trachea, lung, spleen |
| | | Nasal wash | *Collect whole head and |
| | | Throat swab | thoracic pluck |
| | | Lung tissue (2 × sample) | |
| | | For Histology: Nasal cavity, | |
| | | trachea, lung, spleen | |
| | | *collect whole head and | |
| | | thoracic pluck | |
| 11 (4 DPC) | 6 Hamsters, | (Clinical observations (scored) | 6 × Throat swabs-‘media tube’ |
| | Group 2 | and temperature checked | |
| | | twice daily) | |
| | | Group 2: Throat swab | |
| 13 (6 DPC) | 6 Hamsters, | (Clinical observations (scored) | 6 × Throat swab-‘media tube’ |
| | Group 2 | and temperature checked | |
| | | twice daily) | |
| | | Group 2: Throat swab | |
| 14 (7 DPC) | 15 Hamsters, | (Clinical observations (scored) | 15 × 400 ul Nasal Wash −1.5 ml sarstedt |
| | groups 1, 2 | and temperature checked | 15 × Throat Swab-‘Media tube’ |
| | and 3 | twice daily) | 15 × Lung in 1.5 ml sarstedt |
| | | Terminate 15 animals, groups | 15 × Lung in RNAlater tube |
| | | 1, 2 and 3 | Histology: Nasal cavity*, |
| | | Collect, per animal: | trachea, lung, spleen |
| | | Nasal wash | *Collect whole head and |
| | | Throat swab | thoracic pluck |
| | | Lung tissue (2 samples) | |
| | | For Histology: Nasal cavity, | |
| | | trachea, lung, spleen | |
| | | *collect whole head and | |
| | | thoracic pluck | |

TABLE 3
study design summary

| Day Post | | | | | |
|---|---|---|---|---|---|
| Challenge | Sentinel | Control | Treatment | Control | Treatment |
| −7 | Dose, PBS | Dose, PBS | Dose, | Dose, PBS | Dose, |
| | | | Neumifil | | Neumifil |
| −6 | | | | | |
| −5 | | | | | |
| −4 | | | | | |
| −3 | Dose, PBS | Dose, PBS | Dose, | Dose, PBS | Dose, |
| | | | Neumifil | | Neumifil |
| −2 | | | | | |
| −1 | Dose, PBS | Dose, PBS | Dose, | Dose, PBS | Dose, |
| | | | Neumifil | | Neumifil |
| 0 | | Challenge | Challenge | Challenge | Challenge |
| | | 5E+04/PFU | 5E+04/PFU | 1E+02/PFU | 1E+02/PFU |
| 1 | | | | | |
| 2 | | | | | |
| 3 | | | | Cull | Cull |
| 4 | | | | | |
| 5 | | | | | |
| 6 | | | | | |
| 7 | Cull | Cull | Cull | | |

Results

TABLE 4
clinical observations for group 2 (PBS) and 3 (Neumifil) following challenge with 5E+04 PFU SARS-CoV-2.

| | Clinical Observations, Days Post Challenge | | | | | | | | | | | | | | |
|---|---|---|---|---|---|---|---|---|---|---|---|---|---|---|---|
| Animal | 0.0 | 0.25 | 1.00 | 1.25 | 1.98 | 2.25 | 2.96 | 3.26 | 3.96 | 4.25 | 4.96 | 5.25 | 5.96 | 6.25 | 6.93 |
| 2-1 | H | H | H | H | H | H | H | H | H | H | H | R, P | R, P | R, P | R, P |
| 2-2 | H | H | H | H | H | H | H | H | H | H | Lb | Lb, R | Lb, R, | Lb, R, | R |
| | | | | | | | | | | | | | P | P | |
| 2-3 | H | H | H | H | H | H | H | H | A | A | A, R, | A, R, | Lb, R, | R, P | R, P |
| | | | | | | | | | | | P | P | P | | |
| 2-4 | H | H | H | H | H | H | H | H | H | H | P | P | H | R, P, | R, P |
| | | | | | | | | | | | | | | D | |
| 2-5 | H | H | H | H | H | H | H | H | P | P | Lb, P | Lb, P | H | R, P | R, P |
| 2-6 | H | H | H | H | H | H | H | H | H | P | Lb, R, | Lb, R, | Lb, R, | R, P, | R, P |
| | | | | | | | | | | | P | P | P | D | |
| 3-1 | H | H | H | H | H | H | H | H | H | H | Lb | Lb, P, | Lb, R | R | R |
| | | | | | | | | | | | | R | | | |
| 3-2 | H | H | H | H | H | H | H | H | H | H | H | H | R, P | R | R, P |
| 3-3 | H | H | H | H | H | H | H | H | H | H | H | H | H | R | H |
| 3-4 | H | H | H | H | H | H | H | H | H | H | H | H | H | R | H |
| 3-5 | H | H | H | H | H | H | H | H | H | H | H | H | H | H | H |
| 3-6 | H | H | H | H | H | H | H | H | H | H | P | H | R, P, D | R, P | R |

H = Healthy,
A = Arched/Hunched,
R = Ruffled fur,
Lb = Laboured breathing,
P = Pinched/Wasp waisted,
D = Dehydrated not drinking
Clinical observations were made twice-daily from 7 days prior to challenge onwards. No clinical symptoms were observed in any group prior to challenge. Note also that in the Neumifil treated group, the average number of symptoms (A, R, Lb, P and/or D) is generally lower than in the control group. Moreover there are fewer instances of Lb in the Neumifil treated group.
Additional results are presented in FIG. 7. As compared to the control group(s), the Neumifil treated group (group 3) exhibited fewer total clinical observations

### EXAMPLE 4

Angiotensin-converting enzyme 2 (ACE2) plays a major role in SARS-CoV-2 recognition, binding, fusion and entry into host cells [1]. Glycans, including sialic acid, may also be important in this interaction. There are eight glycosylation sites within the ACE2 receptor, three of which (N90, N322 and N546) may play a critical role in the interaction with SARS-CoV-2 Spike. Glycans on the SARS-CoV-2 Spike may also modulate the conformation of the Spike's receptor binding domain (RBD), which is responsible for ACE2 recognition and binding. Deletion of these glycans significantly reduces ACE2 binding.

Multiple new variants of SARS-CoV-2 have emerged and are circulating globally [1,2]. Of particular concern are the B.1.1.7 and B.1525 variants identified in the UK, the B.1.351 South African variant and the P.1 Brazil variant. The B.1.1.7 UK (Kent) virus is characterized by the HV69-70 deletion and N501Y in the Spike protein which increases transmissibility and may be associated with increased risk of death. The B.1.351 S. African variant shares some mutations with B.1.1.7 alongside additional differences, including the potential vaccine escape mutation E484K. The Brazil variant, P.1 is spreading to multiple countries and is associated with reinfections. P.1 also includes E484K, in addition to the N501Y mutation linked to increased infectivity of the UK variant. A further variant, B1525, has been identified in the UK and contains the important E484K mutation alongside the Kent B.1.1.7 mutations.

Aims: 1) To determine whether Neumifil interacts with SARS-CoV-2 Spike 51 protein and whether the affinity is affected by new variant mutations. 2) To determine whether Neumifil interacts with human ACE2.

Methods:

Table 1 summarizes the variant 51 Spike sequence information provided by the manufacturers. Each of the Spike and ACE2 proteins were recombinantly expressed in HEK293 cells.

TABLE 5
Spike S1 protein variants used in the study with corresponding
accession codes and mutations.

| Spike Variant | Product sequence information |
|---|---|
| Wuhan | Wuhan-Hu-1 (Dec 2019 isolate). Accession code: |
| | QHD43416.1 |
| B.1.351 (S. | K417N, E484K, N501Y, D614G in Wuhan- |
| African) | Hu-1 background |
| B.1.1.7 (UK | HV69-70 deletion, Y144 deletion, N501Y, A570D, |
| (Kent)) | D614G, P681H in Wuhan-Hu-1 background |

EC50 (half-maximal effective concentration) values were determined by ELISA. Spike or ACE2 proteins were immobilized overnight at 4° C. on a high-binding ELISA plate at a concentration of 1 μg/mL. The wells were then incubated for 1.5 h with Neumifil (3-fold dilution series: 29160, 9720, 3240, 1080, 360, 120, 40, 0 ng/mL) in triplicate. Immunodetection of Neumifil binding was performed by incubation with rabbit anti-Neumifil (1 h), followed by HRP-labelled anti-rabbit IgG (1 h) and TMB substrate development. Binding curves were analysed using 4 parameter logistic (4PL) curve fitting to determine the inflection point (EC50).

Results:

As shown in FIG. 8, Neumifil binds to the original Spike protein (consisting of the Wuhan-Hu-1 Dec. 2019 isolate sequence) with an EC50 of 174 ng/mL. The binding profiles and EC50s for the variants indicate that the affinity is not significantly affected by the mutations present in the South African and UK (Kent) sequences. FIG. 9 shows Neumifil binding to ACE2 with an EC50 of 235 ng/ml.

### REFERENCES

1. Understanding variants of SARS-CoV-2 (2021) The Lancet World Report 397(102), P462

2. McNally, A. (2021) BMJ 372, 504

1. A method of treating or preventing a Coronavirus infection and/or a disease or condition caused or contributed by a Coronavirus, said method comprising administering a glycan binding molecule to a subject in need thereof.

2. The method of claim 1, wherein the disease or condition caused or contributed by a Coronavirus is COVID-19 or SARS or MERS.

3. The method of claim 1, wherein the treatment of a Coronavirus infection and/or a disease or condition caused or contributed by a Coronavirus comprises the treatment of one or more of the symptoms associated with the Coronavirus infection, disease or condition.

4. The method of claim 3, wherein the symptom(s) is/are a continuous cough and/or a fever and/or a change/loss in/of taste/smell.

5. The method of claim 1, wherein the glycan binding molecule comprises a carbohydrate binding module (CBM).

6. The method of claim 5, wherein the CBM is selected from the group consisting of:
(i) A Family 40 CBM; and
(ii) A Family 32 CBM.

7. The method of claim 6, wherein the CBM is selected from the group consisting of:
(i) a Clostridium perfringens CBM32 (CpCBM32);
(ii) a Streptococcus pneumoniae CBM40 (SpCBM40);
(iii) a Vibrio cholerae CBM40 (VcCBM40);
(iv) a Vibrio cholerae NanH sialidase CBM;
(v) a Vibrio cholerae NanH sialidase CBM sialic acid binding fragment thereof
(vi) a Streptococcus pneumoniae nanA sialidase CBM; and
(vii) a Streptococcus pneumoniae nanA sialidase CBM sialic acid binding fragment thereof.

8. The method of claim 1, wherein the Coronavirus is SARS-CoV-2.

9. The method of claim 8, wherein the Coronavirus is a SARS-CoV-2 variant.

10. The method of claim 9, wherein the SARS-CoV-2 variant comprises a mutation within the spike protein, the mutation being an amino acid change relative to the amino acid sequence of the spike protein of the Wuhan-Hu-1 isolate with accession codes: QHD43416.1/YP_009724390.1.

11. The method of claim 10, wherein the variant comprises one or more of the following spike protein mutations:
(i) HV69-70 deletion; and/or
(ii) N501Y; and/or
(iii) E484K.

12. The method of claim 9 wherein the SARS-CoV-2 variant is the B.1.1.7 variant and/or the B.1525 variant and/or the B.1.351 variant.

13. (canceled)

14. The method of claim 1, wherein the glycan binding molecule is a CBM32, and the disease or condition caused or contributed by a Coronavirus is COVID-19, a disease or condition caused or contributed by SARS-CoV-2 and/or a symptom of COVID-19.

15. The method of claim 14, wherein the CBM32 comprises the amino acid sequence of SEQ ID NO: 1:
AIIETAIPQSEMTASATSEEGQDPASSAIDGNTNTMWHTKWNGSDALPQS
LSVNLGSSRKVSSIAITPRTSGNNGFITKYEIHAINNGVETLVAEGTWEE
NNLVKTVTFDSPIDAEEIKITAIQGVGGFASIAELNVYE
or a glycan/carbohydrate binding fragment thereof.

16. A method of treating or preventing:
a Coronavirus infection, a disease or condition caused or contributed by a Coronavirus; or
(ii) COVID-19, a disease or condition caused or contributed by SARS-CoV-2 and/or a symptom of COVID-19;
said method comprising administering a CBM40 to a subject in need thereof.

17. The method of claim 16, wherein the CBM40 comprises the amino acid sequence of SEQ ID NO: 4:
ALFDYNATGDTEEDSPAKQGWMQDNTNNGSGVLTNADGMPAWLVQGIGG
RAQWTYSLSTNQHAQASSFGWRMTTEMKVLSGGMITNYYANGTQRVLPI
ISLDSSGNLVVEFEGQTGRTVLATGTAATEYHKFELVFLPGSNPSASFY
FDGKLIRDNIQPTASKQNMIVWGNGSSNTDGVAAYRDIKFEIQGD
or
SEQ ID NO: 6
VIEKEDVETNASNGQRVDLSSELDKLKKLENATVHMEFKPDAKAPAFYN
LFSVSSATKKDEYFTMAVYNNTATLEGRGSDGKQFYNNYNDAPLKVKPG
QWNSVTFTVEKPTAELPKGRVRLYVNGVLSRTSLRSGNFIKDMPDVTHV
QIGATKRANNTVWGSNLQIRNLTVYNRALTPEEVQKRS
or a glycan/sialic acid binding fragment of either.

18. (canceled)

19. The method of claim 5, wherein the CBM comprises a modified CBM comprising a wild type CBM sequence which has been modified to include one or more mutations.

20. The method of claim 19, wherein the modified CBM comprises the sequence of SEQ ID NO: 17:
GAMVIEKEDVETNASNGQRVDLSSELDKLKKLENATVHMEFKPDPKAPA
FYNLFSVSSATKKDEYFTMAVYNNTATLEGRGSDGKQFYNNYNDAPLKV
KPGQWNSVTFTVEKPTAELPKGRARLYVNGGLSRTSLRSGNFIKDMPDV
THVQIGATKRANNTVWGSNLQIRNLTVYNRALTPEEVOKRSGGGSGVIE
KEDVETNASNGQRVDLSSELDKLKKLENATVHMEFKPDPKAPAFYNLFS
VSSATKKDEYFTMAVYNNTATLEGRGSDGKQFYNNYNDAPLKVKPGQWN
SVTFTVEKPTAELPKGRARLYVNGGLSRTSLRSGNFIKDMPDVTHVQIG
ATKRANNTVWGSNLQIRNLTVYNRALTPEEVQKRSGGSLGVPDFESDWF
DVSSNSLYTLSHGLQRSPRRVVVEFARSSSPSTWNIVMPSYFNDGGHKG
SGAQVEVGSLNIKLGTGAAVWGTGYFGGIDNSATTRFATGYYRVRAWI
or a glycan acid binding fragment thereof.

21. A method of treating or preventing:
a Coronavirus infection, a disease or condition caused or contributed by a Coronavirus and/or a symptom of a Coronavirus infection/disease; or
(ii) COVID-19, a disease or condition caused or contributed by SARS-CoV-2 and/or a symptom of COVID-19;
said method comprising administering a glycan acid binding molecule to a subject in need thereof, wherein the glycan binding molecule is selected from the group consisting of:
Cp2CBM32TD (comprising, consisting essentially of or consisting of: 2 CBMs (CBM32s) from Clostridium perfringens fused to a trimerisation domain);
(ii) Sp2CBM40TD (comprising, consisting essentially of or consisting of: 2 CBMs (CBM40s) from Streptococcus pneumoniae fused to a trimerisation domain;
(iii) Vc2CBM40TD (comprising, consisting essentially of or consisting of: 2 CBMs (CBM40s) from Vibrio cholerae fused to a trimerisation domain; and
(iv) Vc4CBM (comprising, consisting essentially of or consisting of: 4 CBMs (CBM40s) from Vibrio cholerae).
